(19) United States
(12) Patent Application Publication
(10) Pub. No.: US 2021/0262032 A1
(43) Pub. Date: Aug. 26, 2021

(54) DETERMINING RESPONDERS TO INFLAMMATION TREATMENT

(71) Applicants: RAMBAM MED-TECH LTD., Haifa (IL); TECHNION RESEARCH & DEVELOPMENT FOUNDATION LIMITED, Haifa (IL)

(72) Inventors: Yehuda CHOWERS, Tel Aviv (IL); Shai SHEN-ORR, Karkur (IL); Shiran VAINBERG, Tirat Carmel (IL); Elina STAROSVETSKY, Yokneam Illit (IL); Sigal PRESSMAN, Pardes Hanna (IL); Alexandra BLATT, Haifa (IL)

(21) Appl. No.: 17/260,428

(22) PCT Filed: Jul. 15, 2019
(86) PCT No.: PCT/IL2019/050796

§ 371 Date: Jan. 14, 2021

Related U.S. Application Data
(60) Provisional application No. 62/698,185, filed on Jul. 15, 2018.

(51) Int. Cl. C12Q 1/6883; C07K 16/28; A61K 31/661; G01N 33/92

(52) U.S. Cl. CPC C12Q 1/6883; C07K 16/2839; A61K 31/661; G01N 33/92; C12Q 2600/106; C12Q 2600/158; G01N 2800/7095; G01N 2405/04; C07K 2317/24; C07K 2317/76

(57) ABSTRACT

Methods of determining suitability of a subject to treatment with an agent that reduces localized inflammation and for converting an unsuitable subject to a suitable one are provided. Kits comprising molecules for doing same are also provided.

### CROSS-REFERENCE TO RELATED APPLICATIONS

This application claims the benefit of priority to U.S. Provisional Patent Application No. 62/698,185, filed Jul. 15, 2018, entitled “DETERMINING RESPONDERS TO INFLAMMATORY BOWEL DISEASE TREATMENT,” the contents of which are incorporated herein by reference in their entirety.

### FIELD OF INVENTION

The present invention is in the field of therapeutic diagnostics and inflammatory bowel disease treatment.

### SUMMARY OF THE INVENTION

The present invention provides methods of determining suitability of a subject to treatment with a therapeutic agent that reduces localized inflammation and for converting an unsuitable subject into a suitable one.

According to a first aspect, there is provided a method of determining the suitability of a subject in need thereof to be treated with a therapeutic agent that reduces localized inflammation, the method comprising:
a. providing a sample from the subject;
b. measuring in the sample at least one of:
   i. expression of lysophosphatidic acid (LPA) and
   ii. expression of at least one molecule selected from SLC22A4, METTL9, AGPAT3, MBOAT2, ATX and CREB1; and
c. determining the suitability of the subject for treatment according to the expression of the LPA, the expression of the at least one molecule or both, wherein expression beyond a predetermined threshold qualifies the subject for treatment with the therapeutic agent and expression within the predetermined threshold disqualifies the subject for treatment with the therapeutic agent, thereby determining the suitability of a subject to be treated with a therapeutic agent that reduces localized inflammation.

According to another aspect, there is provided a method of determining the suitability of a subject in need thereof to be treated with a therapeutic agent that reduces localized inflammation, the method comprising:
a. providing a sample from the subject;
b. measuring in the sample at least one of:
   i. expression of lysophosphatidic acid (LPA) and
   ii. expression of at least one molecule that regulates LPA expression; and
c. determining the suitability of the subject for treatment according to the expression of the LPA, the expression of the at least one molecule or both, wherein expression beyond a predetermined threshold qualifies the subject for treatment with the therapeutic agent and expression within the predetermined threshold disqualifies the subject for treatment with the therapeutic agent,
thereby determining the suitability of a subject to be treated with a therapeutic agent that reduces localized inflammation.

According to another aspect, there is provided a method of inducing a subject unsuitable to be treated with a therapeutic agent that reduces localized inflammation to be suitable to be treated with the therapeutic agent, comprising, increasing LPA levels or activity in the subject, thereby inducing the subject unsuitable to be treated to be suitable to be treated.

According to another aspect, there is provided a method of treating a subject unsuitable for treatment with a therapeutic agent that reduces localized inflammation, comprising:
a. increasing LPA levels or activity in the subject, and
b. administering the therapeutic agent that reduces localized inflammation,
thereby treating a subject unsuitable for treatment with a therapeutic agent that reduces localized inflammation.

According to another aspect, there is provided a method of reducing secretion of a pro-inflammatory cytokine from a cell, the method comprising contacting the cell with an anti-integrin blocking antibody and LPA, thereby reducing secretion of a pro-inflammatory cytokine from a cell.

According to another aspect, there is provided a method of treating inflammation in a subject, the method comprising administering to the subject an anti-integrin blocking antibody and increasing LPA levels or function in the subject, thereby treating inflammation in a subject.

According to another aspect, there is provided a pharmaceutical composition comprising an anti-integrin blocking antibody and an agent that increases LPA levels or function.

According to another aspect, there is provided a kit comprising at least 2 detection molecules selected from: a detection molecule specific to ATX, a detection molecule specific to CREB1, a detection molecule specific to AGPAT3, a detection molecule specific to SLC22A4, a detection molecule specific to METTL9 and a detection molecule specific to MBOAT2.

According to another aspect, there is provided a kit comprising an anti-integrin blocking antibody and an agent that increases LPA levels, function or both.

According to some embodiments, the subject suffers from inflammatory bowel disease (IBD).

According to some embodiments, the IBD comprises colitis, ulcerative colitis, immune checkpoint-induced cloitis and Crohn's disease.

According to some embodiments, the subject is naïve to treatment, or has received first-line treatment.

According to some embodiments, the sample is a peripheral blood sample or a sample from the gut.

According to some embodiments, measuring expression comprises measuring mRNA expression, protein expression or both.

According to some embodiments, the molecule that regulates LPA expression is a molecule that regulates LPA synthesis.

According to some embodiments, the at least one molecule that regulates LPA expression upregulates LPA expression, the sample is from peripheral blood and the subject is suitable for treatment if expression of the molecule is above the predetermined threshold or wherein the at least one molecule that regulates LPA expression down-regulates LPA expression, the sample is from peripheral blood and wherein the subject is suitable for treatment if expression of the molecule is below the predetermined threshold.

According to some embodiments, the at least one molecule that regulates LPA expression upregulates LPA expression, the sample is a gut sample and the subject is suitable for treatment if expression of the molecule is below the predetermined threshold or wherein the at least one molecule that regulates LPA expression down-regulates LPA expression, the sample is a gut sample and wherein the subject is suitable for treatment if expression of the molecule is above the predetermined threshold.

According to some embodiments, the molecule that regulates LPA synthesis is selected from the group consisting of AGPAT3, MBOAT2, ENPP2 (ATX), and CREB1.

According to some embodiments, the method of the invention comprises measuring in the sample expression of AGPAT3, MBOAT2, ATX and CREB1.

According to some embodiments, the subject is suitable for treatment if expression in blood of at least one of AGPAT3, SLC22A4, METTL9 and MBOAT2 is below the predetermined threshold, expression in blood of at least one of ATX and CREB1 is above the predetermined threshold, or both.

According to some embodiments, the subject is suitable for treatment if expression in a gut sample of at least one of AGPAT3, SLC22A4, METTL9 and MBOAT2 is above the predetermined threshold, expression in a gut sample of at least one of ATX and CREB1 is below the predetermined threshold, or both.

According to some embodiments, the subject is suitable for treatment if expression of LPA in a gut sample is below the predetermined threshold or the expression of LPA in peripheral blood is above the predetermined threshold.

According to some embodiments, the method of the invention further comprises measuring monocyte abundance in the sample and wherein monocyte numbers below a predetermined threshold is indicative of suitability to be treated. According to some embodiments, expression of AGAPT3, MBOAT2 and ATX are measured.

According to some embodiments, the method of the invention further comprises:
d. administering the therapeutic agent that reduces localized inflammation to the suitable subject.

According to some embodiments, a subject unsuitable to be treated with a therapeutic agent that reduces localized inflammation is not responsive to treatment with the therapeutic agent.

According to some embodiments, the subject suffers from IBD.

According to some embodiments, increasing LPA levels or function in the subject comprises administering an agent that increases LPA levels or function in the subject.

According to some embodiments, the increasing LPA levels or function in the subject comprises increasing LPA levels or function in peripheral blood of the subject.

According to some embodiments, increases LPA levels or function in the peripheral blood comprises decreasing LPA levels or function in a mucosa of the subject. According to some embodiments, wherein the mucosa is gut mucosa.

According to some embodiments, decreasing LPA levels in the gut mucosa of the subject, comprises decreasing expression or activity of at least one molecule that increases LPA levels, increasing expression or activity of at least one molecule that decreases LPA level, blocking LPA binding to a gut LPA receptor, or a combination thereof.

According to some embodiments, increasing LPA levels comprises increasing expression or activity of at least one molecule that increases LPA levels, decreasing expression or activity of at least one molecule that decreases LPA levels or both.

According to some embodiments, the increasing LPA levels comprises administering to the subject LPA or an LPA precursor.

According to some embodiments, the LPA precursor is lysophosphatidylcholine (LPC).

According to some embodiments, the at least one molecule that increases LPA is ATX, CREB1 or both.

According to some embodiments, the at least one molecule that decreases LPA levels is AGPAT3, MBOAT2, SLC22A4, METTL9 or a combination thereof.

According to some embodiments, decreasing activity of at least one molecule that increases LPA levels comprising administering an antagonist or inhibitor of ATX, CREB1 or both and increasing activity of at least one molecule that decreases LPA levels comprises administering an agonist or activator of AGPAT3, MBOAT2, SLC22A4, METTL9 or a combination thereof.

According to some embodiments, increasing activity of at least one molecule that increases LPA levels comprising administering an agonist or activator of ATX, CREB1 or both and decreasing activity of at least one molecule that decreases LPA levels comprises administering an antagonist or inhibitor of AGPAT3, MBOAT2, SLC22A4, METTL9 or a combination thereof.

According to some embodiments, increasing LPA activity comprises administering an LPA receptor agonist.

According to some embodiments, the method of the invention further comprises administering the therapeutic agent that reduces localized inflammation to the converted subject.

According to some embodiments, reducing localized inflammation comprises inhibiting cell migration. According to some embodiments, the cell migration is immune cell migration.

According to some embodiments, the inflammation is immune-mediated inflammation.

According to some embodiments, the localized is localized within a tissue. According to some embodiments, the tissue comprises mucosa.

According to some embodiments, the therapeutic agent is a blocking antibody.

According to some embodiments, the blocking antibody is selected from an anti-integrin blocking antibody and an anti-pro-inflammatory cytokine blocking antibody.

According to some embodiments, the anti-integrin blocking antibody is selected from an anti-ITGA4/B7 blocking antibody, an anti-ITGA4 blocking antibody and an anti-ITGB7 blocking antibody.

According to some embodiments, the anti-ITGA4/B7 blocking antibody is Vedolizumab.

According to some embodiments, the anti-ITGB7 blocking antibody is Etrolizumab.

According to some embodiments, the pro-inflammatory cytokine is TNFα.

According to some embodiments, a kit of the invention consists of the detection molecule specific to ATX, the detection molecule specific to CREB1, the detection molecule specific to AGPAT3 and the detection molecule specific to MBOAT2.

According to some embodiments, a kit of the invention further comprises a detection molecule specific to LPA.

According to some embodiments, the detection molecule detects mRNA or protein.

According to some embodiments, the detection molecule is an antibody, a pair of PCR primers or a nucleic acid sequence that hybridizes to the mRNA.

According to some embodiments, a kit of the invention is for determining the suitability of a subject in need thereof to be treated with a therapeutic agent that reduces localized inflammation.

According to some embodiments, a kit of the invention further comprises a therapeutic agent that reduces localized inflammation.

According to some embodiments, a kit of the invention is for determining suitability for treatment with a therapeutic agent that reduces localized inflammation and treatment of IBD.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-B: Gene expression processing and batch correction. Heatmaps of log 2 gene expression of 75% of the genes exhibiting higher variance before (1A) and after batch correction (1B). Log 2 expression is represented by color key. Side bars describe classification of samples by response status, batch and time post first treatment

FIGS. 2A-C: Responding patients show increased estimated proportions of CD4 T cell subsets by deconvolution, which were significantly higher compared to non-responders 14 weeks post-treatment. (2A) Heatmap of scaled scores of cell frequency pre-treatment and 14 weeks post treatment in responders. Scaled scores are represented by color key. Top bar describes p. value of paired t-test. Side bar describes the PCA based distance between V1V3 within patient, classification of samples by response status, batch and time post first treatment. (2B) Comparison of the increased CD4 subsets between responders and non-responders 14 weeks post-treatment. (2C) Dynamics of the CD4 subsets in responding and non-responding patients

FIG. 3: Responding patients have reduced estimated proportion score of Tregs in intestinal tissue while non-responders do not show abundance change. Deconvolved estimated Tregs proportion scores in intestinal tissue and peripheral blood at baseline and in different time points post-treatment in responding and non-responding patients were evaluated usind xCell.

FIGS. 4A-B. Accounting for cells, unmasks differential regulation. (4A) Volcano plot of standard gene expression differential analysis and (4B) cell-centered deconvolution of responding patients between visit 1 (0 w) and visit 3 (14 w) in peripheral blood using linear mixed effects model, comparing FDR versus estimates. Corrected for multiple comparisons was performed using BH FDR.

FIG. 5. Responders and non-responders have similar expression of the drug target—Integrin a4/b7 genes in blood. Pre (GX) and post adjustment (AG) expression of the integrin alpha4 and beta7 chains pre-treatment (V1) and 2 w (V2) and 14 w (V3) post treatment in peripheral blood. Statistical significance was calculated by Wilcoxon test.

FIGS. 6A-C. Responders present changes in integrin downstream signaling following therapy. (6A) Heatmap presenting column scaled log 2 expression values of the differentially expressed integrin downstream signaling genes (p<0.05, paired T test). Top bar describes classification by data-type (pre and post gene expression adjustment). Side bar describes classification of samples by visit. (6B) PCA of differentially expressed integrin related genes between visits. (6C) Responders ordering according to PCA based distance of integrin related.

FIG. 7. Integrin-associated responders dynamics. The heatmap represents column scaled log 2 expression of highly correlated genes with absolute spearman correlation coefficient above 0.75, that were also differentially expressed between visits in responders (FDR<0.05, limma R package).

FIGS. 8A-B. Responders integrin related genes that show differential expression compared to non-responders 14 weeks post first treatment. (8A) A heatmap representing row scaled log 2 expression of differentially expressed genes between responders and non-responders 14 weeks post first treatment based on responders dynamics (FDR<0.1, limma R package). (8B) Network propagation of known interacting proteins using ConcensusPathDB.

FIGS. 9A-G. Responders present increased LPA synthesis capacity in peripheral blood indicating enhanced migratory potential pre-treatment. (9A-B) Boxplot showing LPA related log 2 mRNA expression as measured in peripheral blood of a (9A) first cohort and a (9B) second cohort of responding (R) IBD patients, non-responding (NR) IBD patients, and healthy controls (HC) prior to initiation of Vedolizumab therapy. (9C) ROC curve of classifier of vedolizumab response at baseline based on binomial logistic regression model for each gene individually and for combining the four differentially expressed LPA related genes in peripheral blood. Calculated AUC for the four combined is 0.93 (95% 10-fold cross validation CI 0.83-1.00). (9D) Diagram of LPA regulation. (9E-F) Line graphs of estimated sensitivity and specificity of prediction of Vedolizumab response based on expression of MBOAT, ATX and AGPAT3 and monocyte abundance in blood for (9E) the primary cohort and (9F) the validation cohort. (9G) Boxplots showing MBOAT2 expression values pre-treatment from intestines of responders and non-responders to Vedolizumab and Etrolizumab from two publicly available data-sets.

FIGS. 10A-C. Responders present increased serum LPA level. (10A) Boxplot showing serum LPA protein level of 14 and 16 responding (R) and non-responding (NR) patients respectively, prior to initiation of Vedolizumab therapy. Serum LPA concentrations were measured using ELISA. (10B) ROC curve of classifier of vedolizumab response at baseline based on binomial logistic regression model for LPA levels. (10C) Boxplot showing serum LPA level of 14 and 11 responding (R) and non-responding (NR) patients respectively, prior to initiation of Infliximab therapy. Serum LPA concentrations were measured using ELISA.

FIGS. 11A-B: Anti-inflammatory synergistic effect between Vedolizumab and LPA in-Vitro. (11A-B) Bar charts showing inflammatory cytokine expression including (11A) TNFα and (11B) IL-1β post incubation with Vedolizumab (600 μg/ml), LPA (1 μM) and co-incubation of the two substances, using qPCR for quantification. Un-treated whole blood basal expression served as control and values were expressed as fold-induction over control. Data were pooled from three independent experiments. Significance was calculated using Wilcoxon-test (two-sided; μ=1)

### DETAILED DESCRIPTION OF THE INVENTION

The present invention, in some embodiments, provides methods of determining the suitability of a subject to a treatment with an anti-localized inflammation agent and well as converting unsuitable subjects to suitable subjects. The present invention further concerns a method of determining suitability for treatment and treating subjects with an anti-localized inflammation therapeutic agent. Methods of reducing inflammatory cytokine secretion from a cell and treating inflammation in a subject are also provided. Kits for performing the methods of the invention are also provided.

The present invention is based on the surprising finding that patients suffering from inflammatory bowel disease (IBD) who do not respond to treatment with an ITGA4/B7 and ITGB7 blocking antibodies have lower levels of circulating lysophosphatidic acid (LPA) before treatment than do patients that do respond to the treatment. Further, it was discovered that though LPA protein levels can distinguish between future responders and non-responders, unexpectedly, the expression levels of six genes involved in LPA synthesis are even better predictors of clinical outcome. These results are surprising as it has been suggested that inhibition of LPA synthesis might be a potential treatment for inflammatory diseases, and specifically for IBD (Thirunavukkarasu et al., J. Pharmacol Exp. Ther., 2016, 359(1):207-214). Conversely, the invention provides a method of treating IBD that comprises increasing LPA levels in the subject. It was further surprising found that combination of LPA and an ITGA4/B7 blocking antibody decreases pro-inflammatory cytokine secretion from blood cells in vitro. This further, supports use of LPA to treat inflammation, and in particular in combination with an integrin blocking antibody.

By a first aspect, there is provided a method of determining the suitability of a subject in need thereof to be treated with a therapeutic agent, the method comprising:
a. providing a sample from the subject;
b. measuring in the sample at least one of;
   i. expression of LPA, and
   ii. expression of at least one molecule that regulates LPA expression; and
c. determining the suitability of the subject for treatment according to the expression of LPA, the expression of the at least one molecule or both, wherein expression beyond a predetermined threshold qualifies the subject for treatment with the therapeutic agent and expression within the predetermined threshold disqualifies the subject for treatment with the therapeutic agent, thereby determining the suitability of a subject to be treated with a therapeutic agent.

By another aspect, there is provided a method of determining the suitability of a subject in need thereof to be treated with a therapeutic agent that reduces localized inflammation, the method comprising:
a. providing a sample from the subject;
b. measuring in the sample at least one of:
   i. expression of lysophosphatidic acid (LPA) and
   ii. expression of at least one molecule selected from SLC22A4, METTL9, AGPAT3, MBOAT2, ATX and CREB1; and
c. determining the suitability of said subject for treatment according to the expression of the LPA, the expression of the at least one molecule or both, wherein expression beyond a predetermined threshold qualifies the subject for treatment with the therapeutic agent and expression within the predetermined threshold disqualifies the subject for treatment with satheid therapeutic agent,
thereby determining the suitability of a subject to be treated with a therapeutic agent that reduces localized inflammation.

By another aspect, there is provided a method of determining the suitability of a subject in need thereof to be treated with a therapeutic agent and treating the subject, the method comprising:
a. providing a sample from the subject;
b. measuring in the sample at least one of:
   i. expression of lysophosphatidic acid (LPA);
   ii. expression of at least one molecule selected from SLC22A4, METTL9, AGPAT3, MBOAT2, ATX and CREB1; and
   iii. expression of at least one molecule that regulates LPA expression;
c. determining the suitability of the subject for treatment according to the expression of the LPA, the expression of the at least one molecule or both, wherein expression beyond a predetermined threshold qualifies the subject for treatment with the therapeutic agent and expression within the predetermined threshold disqualifies the subject for treatment with the therapeutic agent; and
d. administering the therapeutic agent that inhibits cell migration to the suitable subject,
   thereby determining the suitability of a subject to be treated with a therapeutic agent and treating the subject.

By another aspect, there is provided a method of inducing a subject unsuitable to be treated with a therapeutic agent to be suitable to be treated with the therapeutic agent, comprising, increasing LPA levels or activity in the subject, thereby inducing the subject unsuitable to be treated to be suitable to be treated.

By another aspect, there is provided a method of converting a subject unsuitable to be treated with a therapeutic agent to be a suitable subject, comprising, increasing ATX and CREB1 levels or activity in the subject and decreasing AGPAT3, SLC22A4, METTL9, and MBOAT2 levels or activity beyond a predetermined threshold, thereby converting the unsuitable subject to a suitable subject.

By another aspect, there is provided a method of treating a subject unsuitable for treatment with a therapeutic agent, comprising:
a. increasing LPA levels or activity in the subject, and
b. administering the therapeutic agent,
   thereby treating a subject unsuitable for treatment.

By another aspect, there is provided a method of treating a subject unsuitable for treatment with a therapeutic agent, comprising:
a. increasing ATX and CREB1 levels or activity and decreasing AGPAT3, SLC22A4, METTL9, and MBOAT2 levels or activity in the subject beyond a predetermined threshold, and
b. administering the therapeutic agent,
   thereby treating a subject unsuitable for treatment.

In some embodiments, the methods of the invention are performed ex-vivo. In some embodiments, the diagnostic aspects of the methods of the invention are performed ex-vivo. It will be understood by a skilled artisan that all steps of the invention that include administering a therapeutic agent to a subject will require in-vivo action of the therapeutic.

In some embodiments, the therapeutic agent is an agent that inhibits cell migration. In some embodiments, the therapeutic agent is an agent that reduces/decreases inflammation. In some embodiments, the inflammation is localized inflammation. In some embodiments, the inflammation is immune mediated inflammation. In some embodiments, the inflammation is innate immunity mediated inflammation. In some embodiments, the inflammation is adaptive immunity mediated inflammation. In some embodiments, reducing localized inflammation comprises inhibiting cell migration. In some embodiments, the inflammation is T cell induced inflammation. In some embodiments, the inflammation is macrophage induced inflammation.

As used herein, “innate immunity” refer to antigen-independent immune response. As used herein “adaptive immunity” refers to antigen-dependent immune response. The two branches of immune response are well known in the art and the cells and signaling molecules that are part of each form of immunity are well known.

In some embodiments, the agent that inhibits cell migration inhibits immune cell migration. In some embodiments, the cell is an immune cell. In some embodiments, the immune cell is a cell of the innate immune system. In some embodiments, the immune cell is a cell of the adaptive immunity system. In some embodiments, the immune cell is a T cell. In some embodiments, the T cell is a CD4 T cell. In some embodiments, the cell is a mucosal cell. In some embodiments, the mucosal cell is a gut mucosal cell. In some embodiments, the cell is a cell of the intestines. In some embodiments, the cell is a gut cell. In some embodiments, the cell is not a gut cell. In some embodiments, the cell is not an intestinal cell. In some embodiments, the cell is a circulating cell. In some embodiments, the cell is a blood cell. In some embodiments, the migration is migration to a mucosa. In some embodiments, the migration is migration to the gut and/or intestines. In some embodiments, the migration is migration to the gut mucosa. In some embodiments, the immune cell is selected from a T cell, a macrophage and a natural killed cell. In some embodiments, the immune cell is a pro-inflammatory immune cell. In some embodiments, the immune cell is a T cell. In some embodiments, the T cell is selected from a T regulatory cell, a T effector cell, a T helper cell, a T cytotoxic cell, a T memory cell, a natural killer T cell and a musical associated invariant T cell. In some embodiments, the T cell is a T regulatory cell. In some embodiments, the T cell is a T cytotoxic cell. In some embodiments, the T cell is a pro-inflammatory T cell. In some embodiments, the T cell is a mucosal associated T cell.

In some embodiments, the therapeutic agent that inhibits cell migration inhibits cell migration to mucosa. In some embodiments, the therapeutic agent that inhibits cell migration inhibits cell migration to the gut/intestines. In some embodiments, the therapeutic agent that inhibits cell migration inhibits cell migration to the gut mucosa. In some embodiments, the therapeutic agent that inhibits migration inhibits migration to cites of inflammation. In some embodiments, the therapeutic agent that inhibits cell migration inhibits integrin function. Integrins are well known in the art and are known to regulate cell adhesion, chemotaxis and migration. Any therapeutic agent that inhibits integrin function and thus cell migration may be the therapeutic agent described herein. In some embodiments, inhibiting integrin function comprises inhibiting the ligand or binding partner of the integrin. Inhibiting function is not limited to directly binding the integrin's activation site, but rather encompasses any mechanism of inhibiting integrin-mediated cell migration. This includes, but is not limited to, binding its activation site, blocking its activation site, blocking dimerization, blocking the ligand or binding partner, altering or inhibiting downstream signaling, altering integrin regulated signaling or transcription. In some embodiments, the integrin is integrin alpha 4 (ITGA4). In some embodiments, the integrin is beta 7 (ITGB7). In some embodiments, the integrin is ITGA4/B7. In some embodiments, the therapeutic agent blocks ITGA4/B7 function. In some embodiments, the therapeutic agent blocks a ligand of ITGA4B7. In some embodiments, the ligand is MAdCAM1.

In some embodiments, the agent that decreases inflammation decreases local inflammation. In some embodiments, an agent that decreases local inflammation also decreases general inflammation. In some embodiments, local is localized within a tissue. In some embodiments, the tissue comprises a mucosa. In some embodiments, the tissue is a mucosa. In some embodiments, the agent that decreases inflammation inhibits pro-inflammatory cytokine function. In some embodiments, the agent that decreases inflammation enhances anti-inflammatory cytokine function. In some embodiments, the agent that decreases inflammation is a pro-inflammatory cytokine antagonist. In some embodiments, the agent that decreases inflammation is an anti-pro-inflammatory cytokine antibody. In some embodiments, the pro-inflammatory cytokine is TNFα. In some embodiments, the pro-inflammatory cytokine is IL-1B. In some embodiments, the agent that decreases inflammation is an anti-TNFα antibody. In some embodiments, the anti-TNFα antibody is Infliximab.

In some embodiments, the agent is selected from an agent that inhibits cell migration and an anti-proinflammatory cytokine agent. In some embodiments, the agent is selected from an anti-integrin agent and an anti-proinflammatory cytokine agent. In some embodiments, the agent is selected from an anti-ITGA4/B7, anti-ITGB7 and an anti-TNFα agent.

In some embodiments, the agent is an antibody. In some embodiments, the agent is a blocking antibody. In some embodiments, the agent is a monoclonal antibody. In some embodiments, the agent is a humanized antibody. In some embodiments, the agent is chimeric antibody. In some embodiments, that agent is an antigen binding fragment. In some embodiments, the agent is an antigen binding fragment of an antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the subject is a mammal. In some embodiments, the subject is a human. In some embodiments, the subject suffers for inflammation. In some embodiments, the subject suffers from inflammation in the bowel. In some embodiments, the subject suffers from inflammatory bowel disease (IBD). In some embodiments, the subject suffers from colitis. In some embodiments, the subject suffers from ulcerative colitis (UC). In some embodiments, colitis is immune checkpoint-induced colitis. In some embodiments, the subject suffers from Crohn's disease (CD). In some embodiments, the subject suffers from Behcet's disease (BD). In some embodiments, the subject suffers from IBD unclassified (IBDU). In some embodiments, IBD comprises colitis, UC and CD. In some embodiments, IBD comprises UC and CD. In some embodiments, IBD comprises UC, CD and BD. In some embodiments, the subject suffers from graft versus host disease (GVHD). In some embodiments, the subject suffers from post-immune checkpoint treatment side effects. In some embodiments, the subject suffers from sarcoidosis. In some embodiments, the subject suffers from a disease treatable by a therapeutic agent that inhibts cell migration. In some embodiments, the subject suffers from a disease treatable by a therapeutic agent that blocks integrin function. In some embodiments, the subject suffers from a disease treatable by a therapeutic agent that blocks ITGA4 function. In some embodiments, the subject suffers from a disease treatable by a therapeutic agent that blocks ITGB7 function. In some embodiments, the subject suffers from a disease treatable by a therapeutic agent that blocks ITGA4/B7 function. In some embodiments, the subject suffers from a Vedolizumab-treatable disease. In some embodiments, the subject suffers from an Etrolizumab-treatable disease. In some embodiments, the subject suffers from a disease treatable by a therapeutic agent that blocks a pro-inflammatory cytokine. In some embodiments, the subject suffers from an Infliuximab-treatable disease.

In some embodiments, the subject is naïve to treatment. In some embodiments, the subject has not been treated with a therapeutic agent that reduces localized inflammation. In some embodiments, the subject has not been treated with a therapeutic agent that inhibits cell migration. In some embodiments, the subject has not been treated with a therapeutic agent that inhibits integrin function. In some embodiments, the subject has not been treated with a therapeutic agent that blocks ITGA4/B7 function. In some embodiments, the subject has not been treated with a therapeutic agent that blocks ITGB7 function. In some embodiments, the subject is naïve to treatment with Vedolizumab and derivatives or generics thereof. In some embodiments, the subject is naïve to treatment with Etrolizumab and derivatives or generics thereof. In some embodiments, the subject has not been treated with a therapeutic agent that blocks a pro-inflammatory cytokine. In some embodiments, the subject has not been treated with a therapeutic agent that blocks TNFα. In some embodiments, the subject is naïve to treatment with Infliximab and derivatives or generics thereof. In some embodiments, the subject has received first-line treatment. A skilled artisan will appreciate that a first-line treatment is dependent on the disease to be treated. For conditions such as IBD the treatment may be with an immune suppressant (thiopurines, methotrexate), TNF-inhibitor, anti p40, or a corticosteroid for non-limiting example.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, a therapeutic agent that blocks integrin function binds to an integrin. In some embodiments, the therapeutic agent binds to a ligand of integrin. In some embodiments, the therapeutic agent is an integrin antagonist. In some embodiments, the therapeutic agent blocks integrin downstream signaling. In some embodiments, the therapeutic agent is an anti-integrin antibody. In some embodiments, the therapeutic agent is an anti-integrin blocking antibody. In some embodiments, the therapeutic agent is a small molecule that binds to and blocks integrin function. In some embodiments, a therapeutic agent that blocks IGTA4/B7 function binds to IGTA4/B7. In some embodiments, the therapeutic agent binds to a ligand of IGTA4/B7. In some embodiments, the therapeutic agent is an IGTA4/B7 antagonist. In some embodiments, the therapeutic agent blocks IGTA4/B7 downstream signaling. In some embodiments, the therapeutic agent is an anti-IGTA4/B7 antibody. In some embodiments, the therapeutic agent is an anti-IGTA4/B7 blocking antibody. In some embodiments, the therapeutic agent is a small molecule that binds to and blocks IGTA4/B7 function. In some embodiments, blocking function comprises blocking binding to its ligand. In some embodiments, blocking function comprises blocking downstream signaling. In some embodiments, blocking function comprises blocking migration of a cell to the bowels. In some embodiments, the therapeutic agent is a monoclonal antibody against IGTA4/B7.

In some embodiments, the therapeutic agent is Natalizumab. In some embodiments, the therapeutic agent is Etrolizumab. In some embodiments, the therapeutic agent is an anti-MAdCAM1 antibody. Other examples of small molecule anti-migration agents include, but are not limited to, anti-CCR9 agents and also SP1 agonists.

In some embodiments, the therapeutic agent is Vedolizumab. In some embodiments, the blocking antibody is Vedolizumab. As used herein, the term “Vedolizumab” refers to a monoclonal anti-IGTA4/B7 antibody that is commercially available. Vedolizumab is sometimes sold under the name Entyvio. In some embodiments, the therapeutic agent is Vedolizumab, an equivalent of Vedolizumab, a derivative of Vedolizumab or a generic of Vedolizumab. In some embodiments, the therapeutic agent has at least one antigen binding domain in common with Vedolizumab. In some embodiments, the therapeutic agent has at least one antigen binding domain that is derived from Vedolizumab.

In some embodiments, the therapeutic agent is Etrolizumab. In some embodiments, the blocking antibody is Etrolizumab. As used herein, the term “Etrolizumab” refers to a monoclonal anti-IGTB7 antibody that is commercially available. In some embodiments, the therapeutic agent is Etrolizumab, an equivalent of Etrolizumab, a derivative of Etrolizumab or a generic of Etrolizumab. In some embodiments, the therapeutic agent has at least one antigen binding domain in common with Etrolizumab. In some embodiments, the therapeutic agent has at least one antigen binding domain that is derived from Etrolizumab.

some embodiments, the therapeutic agent is Infliximab. In some embodiments, the blocking antibody is Infliximab. As used herein, the term “Infliximab” refers to a chimeric monoclonal anti-TNFα antibody that is commercially available. Infliximab is sometimes sold under the name Remicade. In some embodiments, the therapeutic agent is Infliximab, an equivalent of Infliximab, a derivative of Infliximab or a generic of Infliximab. In some embodiments, the therapeutic agent has at least one antigen binding domain in common with Infliximab. In some embodiments, the therapeutic agent has at least one antigen binding domain that is derived from Infliximab.

As used herein, the term “derived from” or “corresponding to” refers to construction of an amino acid sequence based on the knowledge of a sequence using any one of the suitable means known to one skilled in the art, e.g. chemical synthesis in accordance with standard protocols in the art.

In some embodiments, the therapeutic agent alters T cell migration. In some embodiments, the therapeutic agent blocks or inhibits T cell migration. In some embodiments, the migration is to the bowel. In some embodiments, the therapeutic agent alters T cell chemotaxis. In some embodiments, the therapeutic agent induces T cell migration away from the bowel.

In some embodiments, a sample from the subject is provided. In some embodiments, the providing comprises withdrawing a sample from the subject. In some embodiments, the sample is a bodily fluid. Bodily fluids include for example, blood, plasma, urine, lymph, stool, saliva, semen, and breast milk. In some embodiments, the sample is a blood sample. In some embodiments, the sample is any one of blood, urine and saliva. In some embodiments, the sample is plasma. In some embodiments, the blood is peripheral blood. In some embodiments, the sample is a gut sample. Examples of gut samples include, but are not limited to, blood of the gut, stool, and a biopsy. In some embodiments, the sample is not from the intestines. In some embodiments, the providing comprises drawing a blood sample. In some embodiments, the sample is not processed before the measuring. In some embodiments, the sample is processed before the measuring. In some embodiments, intact cells are removed from the sample before the measuring. In some embodiments, protein is extracted from the sample before the measuring. In some embodiments, nucleic acids are extracted from the sample before the measuring.

In some embodiments, the measuring occurs in the sample. Examples of such in situ measuring include for example by ELISA. In some embodiments, the measuring occurs in a composition comprising material extracted from the sample, such as by PCR with extracted nucleic acids. In some embodiments, the measuring expression comprises measuring mRNA expression. In some embodiments, the measuring expression comprises measuring protein expression. In some embodiments, the measuring expression comprises measuring mRNA and protein expression. In some embodiments, the measuring is of expression of LPA. In some embodiments, the measuring is measuring LPA expression. In some embodiments, the LPA is albumen-bound LPA.

In some embodiments, measuring LPA expression comprises measuring free-LPA. In some embodiments, measuring LPA expression comprises measuring albumen-bound LPA. In some embodiments, measuring LPA expression comprises measuring blood LPA levels. In some embodiments, measuring LPA expression comprises measuring peripheral blood LPA. In some embodiments, measuring LPA expression does not comprise measuring intestinal LPA. In some embodiments, measuring LPA expression does not comprise measuring gut LPA.

In some embodiments, the measuring is measuring expression of at least one molecule that regulates LPA expression. In some embodiments, the measuring is measuring expression of a plurality of molecules that regulate LPA expression. In some embodiments, the measuring is measuring expression of at least 1, 2, 3, 4, 5, or 6 molecules that regulates LPA expression. Each possibility represents a separate embodiment of the invention. In some embodiments, the molecule regulates LPA synthesis. In some embodiments, the molecule regulated LPA homeostasis. In some embodiments, the molecule regulates LPA metabolism and/or catabolism. In some embodiments, the molecule regulates LPA stability. In some embodiments, the molecule regulates LPA half-life. In some embodiments, the molecule is an enzyme. In some embodiments, the molecule is a regulatory RNA. In some embodiments, the molecule is a transcription factor.

In some embodiments, the molecule is in peripheral blood and upregulates LPA expression and the subject is suitable for treatment if expression of the molecule is above a predetermined threshold. In some embodiments, the molecule is in peripheral blood and downregulates LPA expression and the subject is suitable for treatment is expression of the molecule is below a predetermined threshold. In some embodiments, the threshold is average expression level in a group of non-responders. In some embodiments, the threshold is the highest or lowest expression in a group of non-responders.

In some embodiments, the molecule is in the gut and upregulates LPA expression and the subject is suitable for treatment if expression of the molecule is below a predetermined threshold. In some embodiments, the molecule is in the gut and downregulates LPA expression and the subject is suitable for treatment is expression of the molecule is above a predetermined threshold. In some embodiments, expression in the gut and peripheral blood have an inverse relationship.

In some embodiments, the at least one molecule that regulates LPA expression or synthesis is selected from the group consisting of AGPAT3, MBOAT2, ATX and CREB1. In some embodiments, molecule is AGPAT3. In some embodiments, molecule is MBOAT2. In some embodiments, molecule is SLC22A4. In some embodiments, molecule is METTL9. AGPAT3 expression, and MBOAT2 expression both decrease LPA expression as they drive the synthesis of a different molecule at the expense of LPA. If levels of AGAT3, SLC22A4, METTL9 and MBOAT2 are below a predetermined threshold in peripheral blood a subject is determined to be suitable for treatment. Similarly, decreasing the levels of AGPAT3, SLC22A4, METTL9 and/or MBOAT2 in peripheral blood can make a subject more suitable for treatment. In some embodiments, the molecule is ATX. In some embodiments, the molecule is CREB1. ATX also known as autotaxin and ENPP2, and is the main enzyme involved in the synthesis of LPA. CREB1 is a transcription factor that increases transcription of ATX and therefore also levels of LPA. If in peripheral blood levels of ATX and/or CREB1 are above a predetermined threshold a subject is determined to be suitable for treatment. Similarly, increasing the levels of ATX and or CREB1 in peripheral blood can make a subject more suitable for treatment. In some embodiments, a subject is suitable for treatment if expression of AGPAT3, SLC22A4, METTL9 and/or MBOAT2 in peripheral blood is below a predetermined threshold. In some embodiments, a subject is suitable for treatment if expression of CREB1 and/or ATX is above a predetermined threshold in peripheral blood. In some embodiments, a subject is suitable for treatment if expression of AGPAT3, SLC22A4, METTL9 and/or MBOAT2 is below a predetermined threshold and expression of CREB1 and/or ATX is above a predetermined threshold in peripheral blood. In some embodiments, a subject is suitable for treatment if expression of AGPAT3, SLC22A4, METTL9 and MBOAT2 is below a predetermined threshold and expression of CREB1 and ATX is above a predetermined threshold in peripheral blood. In some embodiments, each gene must be beyond its threshold expression. In some embodiments, expression values for a gene or protein are standardized or normalized before comparing to a threshold. In some embodiments, the standardizing or normalizing is as compared to a house keeping gene/protein, or to a calibration curve. Standardizing expression levels between subject's is well known in the art, and any method known to a skilled artisan may be used.

If levels of AGAT3, SLC22A4, METTL9 and MBOAT2 are below a predetermined threshold in gut a subject is determined to be unsuitable for treatment. Similarly, increasing the levels of AGPAT3, SLC22A4, METTL9 and/or MBOAT2 in gut can make a subject more suitable for treatment. If in the gut levels of ATX and/or CREB1 are above a predetermined threshold a subject is determined to be unsuitable for treatment. Similarly, decreasing the levels of ATX and or CREB1 in the gut can make a subject more suitable for treatment. In some embodiments, a subject is suitable for treatment if expression of AGPAT3, SLC22A4, METTL9 and/or MBOAT2 in gut is above a predetermined threshold. In some embodiments, a subject is suitable for treatment if expression of CREB1 and/or ATX is below a predetermined threshold in gut. In some embodiments, a subject is suitable for treatment if expression of AGPAT3, SLC22A4, METTL9 and/or MBOAT2 is above a predetermined threshold and expression of CREB1 and/or ATX is below a predetermined threshold in gut. In some embodiments, a subject is suitable for treatment if expression of AGPAT3, SLC22A4, METTL9 and MBOAT2 is above a predetermined threshold and expression of CREB1 and ATX is below a predetermined threshold in gut.

In some embodiments, a subject is suitable for treatment if expression of LPA in gut is below a predetermined threshold. In some embodiments, a subject is suitable for treatment is expression of LPA in peripheral blood is above a predetermined threshold. In some embodiments, gut is a gut sample. In some embodiments, peripheral blood is a peripheral blood sample.

In some embodiments, the threshold is an expression level above a predetermined expression level. In some embodiments, the threshold is a predetermined number of standard deviations above a baseline expression. In some embodiments, the baseline expression is the average expression in non-responders. In some embodiments, the threshold is 0.25, 0.5, 0.75, 1, 1.25, 1.5, 1.75, 2, 2.25, 2.5, 2.75, or 3 standard deviations above a baseline. Each possibility represents a separate embodiment of the invention. In some embodiments, all four genes are measured, and their combined expression must be above a predetermined threshold. In some embodiments, the determining a subject's suitability comprises generating a probability of response value. The probability of response, or probability of response value, is a single number value that defines whether a subject is more likely to respond or not respond. A value of above 0.5 indicates the subject is more likely to respond and should be administered the therapeutic agent. A value below 0.5 indicates the subject is more likely not to respond and should not be administered the therapeutic. A value of 0.5 may be left to the doctor's discretion or may indicate the subject should be given the therapeutic regardless. In some embodiments, the probability response score is determined by weighting the values of each measured gene. In some embodiments, the weighting is done by multiplying by a predetermined coefficient. In some embodiments, the coefficient is determined empirically by expression levels in responding and non-responding patients. In some embodiments, the expression values for a measured gene are log 2 expression values. In some embodiments, the expression values for a measured gene are average expression values. In some embodiments, the expression values for a measured gene are geometric means of the expression values. In some embodiments, the expression values for a measured gene are averages of more than one measuring from a subject. In some embodiments, in order to increase accuracy several measurings, optionally from several samples collected at the same time or different times, are performed. In some embodiments, the probability score is determined by dividing e raised to the sum of each gene's expression times its weighted coefficient by e+1 raised to the same value. In some embodiments, the probability response score is determined using the values in Table 1 and equation 1.

TABLE 1
LPA-relate genes coefficients based on results of
multivariate regression analysis

| Variable | Coefficient | P value |
|---|---|---|
| Intercept (β0) | −8.172 | |
| CREB1 (β1) | 8.64 | 0.057 |
| MBOAT2 (β2) | −3.53 | 0.062 |
| ENPP2 (β3) | 7.159763 | 0.0279 |
| AGPAT3 (β4) | −9.763699 | 0.04791 |

According to the calculated model coefficients, the following formula can be used to predict probability for drug response:

$$P ( probability for response ) = \frac{exp ( β0 + β1 * X 1 + β2 * X 2 + β3 * X 3 + β4 * X 4 )}{1 + exp ( β0 + β1 * X 1 + β2 * X 2 + β3 * X 3 + β4 * X 4 )} \qquad ( Eq . 1 )$$

Where X1, X2, X3, X4 are the log 2 measured peripheral blood gene expression values of CREB1, MBOAT2, ENPP2 and AGPAT3 genes of the patient to be diagnosed. In some embodiments, the determining comprises inputting the measured expressions into equation 1 and wherein a probability value equal to or above 0.5 indicates the subject is suitable for treatment.

In some embodiments, at least one of AGPAT3, MBOAT2, ATX and CREB1 is measured. In some embodiments, at least two of AGPAT3, MBOAT2, ATX and CREB1 are measured. In some embodiments, at least three of AGPAT3, MBOAT2, ATX and CREB1 are measured. In some embodiments, all four of AGPAT3, MBOAT2, ATX and CREB1 are measured. In some embodiments, AGPAT3 and MBOAT2 are measured. In some embodiments, AGPAT3 and ATX are measured. In some embodiments, MBOAT2 and ATX are measured. In some embodiments, MBOAT2 and CREB1 are measured. In some embodiments, ATX and CREB1 are measured. In some embodiments, AGPAT3 and CREB1 are measured. In some embodiments, AGPAT3, MBOAT2 and ATX are measured. In some embodiments, AGPAT3, MBOAT2 and CREB1 are measured. In some embodiments, CREB1, MBOAT2 and ATX are measured. In some embodiments, CREB1, AGPAT3, MBOAT2 and ATX are measured.

In some embodiments, at least one of SLC22A4, METTL9, AGPAT3, MBOAT2, ATX and CREB1 is measured. In some embodiments, at least two of SLC22A4, METTL9, AGPAT3, MBOAT2, ATX and CREB1 are measured. In some embodiments, at least three of SLC22A4, METTL9, AGPAT3, MBOAT2, ATX and CREB1 are measured. In some embodiments, at least four of SLC22A4, METTL9, AGPAT3, MBOAT2, ATX and CREB1 are measured. n some embodiments, at least five of SLC22A4, METTL9, AGPAT3, MBOAT2, ATX and CREB1 are measured. n some embodiments, all of SLC22A4, METTL9, AGPAT3, MBOAT2, ATX and CREB1 are measured.

In some embodiments, mRNA from the genes is measured. In some embodiments, the measuring comprises the step of obtaining nucleic acid molecules from the sample. In some embodiments, the nucleic acids molecules are selected from mRNA molecules, DNA molecules and cDNA molecules. In some embodiments, the cDNA molecules are obtained by reverse transcribing the mRNA molecules. In some embodiments, the expression is determined by measuring mRNA levels of the genes. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995).

Numerous methods are known in the art for measuring expression levels of a one or more gene such as by amplification of nucleic acids (e.g., PCR, isothermal methods, rolling circle methods, etc.) or by quantitative in situ hybridization. Design of primers for amplification of specific genes is well known in the art, and such primers can be found or designed on various websites such as http://bioinfo.ut.ee/primer3-0.4.0/or https://pga.mgh.harvard.edu/primerbank/for example.

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

In some embodiments, protein expression from the genes is measured. In some embodiments, the expression, and the level of expression, of proteins or polypeptides of interest can be detected through immunohistochemical staining of tissue slices or sections. Additionally, proteins/polypeptides of interest may be detected by Western blotting, ELISA or Radioimmunoassay (MA) assays employing protein-specific antibodies.

Alternatively, protein levels can be determined by constructing an antibody microarray in which binding sites comprise immobilized, preferably monoclonal, antibodies specific to a plurality of proteins of interest. Methods for making monoclonal antibodies are well known (see, e.g., Harlow and Lane, 1988, Antibodies: a laboratory manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art.

In some embodiments, other clinical characteristics are considered in determining suitability. A skilled artisan will be aware than when determining drug suitability, a physician may consider a subject's full medical history. Such other clinical characteristics include, but are not limited to, age, BMI, albumin levels, complete blood counts (CBC), and C-reactive protein results.

In some embodiments, the method further comprises measuring abundance of an immune cell in the sample, wherein quantities of the immune cell above a predetermined threshold indicate the subject is suitable to be treated. In some embodiments, the method further comprises measuring abundance of an immune cell in the sample, wherein quantities of the immune cell below a predetermined threshold indicate the subject is suitable to be treated. In some embodiments, the immune cell is a monocyte. In some embodiments, monocyte levels below a predetermined threshold in blood indicate the subject is suitable to be treated. In some embodiments, expression of AGAPT3, MBOAT2 and ATX is combined with abundance of monocytes to determine suitability.

In some embodiments, method further comprises:
d. administering the therapeutic agent to the suitable subject.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of the therapeutic agent to a patient determined to be suitable for treatment. One aspect of the present subject matter provides for rectal administration of the therapeutic agent to a patient determined to be suitable for treatment. Other suitable routes of administration can include parenteral, subcutaneous, oral, rectal, intramuscular, enema, intra-intestinal or intraperitoneal.

The dosage administered will be dependent upon the age, health, and weight of the recipient, kind of concurrent treatment, if any, frequency of treatment, and the nature of the effect desired.

Administration may be directed toward the intestines or toward the blood as is indicated by the therapeutic composition being administered. Agents increasing LPA levels and/or function may be administered to the blood, while agents decreasing LPA levels and/or function may be administered directly to the intestines. In some embodiments, the therapeutic agent is formulated for delivery to the intestines. In some embodiments, the therapeutic agent is in a composition with a targeting motif for the intestines, gut or mucosa. In some embodiments, the therapeutic agent is in a composition configured for oral delivery so as to pass through the stomach to the intestines. Methods of targeting therapeutics, and compositions that target to the gut are well known in the art, and any such method may be employed for delivery of a therapeutic agent that is intended to act in the gut. In some embodiments, administration is targeted to the blood. In some embodiments, administration is targeted away from the intestines.

In some embodiments, a method of the invention converts an unsuitable subject into a suitable subject. As used herein, the term “converting” refers to making subjects that are non-responsive to the therapeutic agent become responsive to the agent. In some embodiments, an unsuitable subject is a subject that does not respond to treatment, and a suitable subject is a subject that responds to treatment. In some embodiments, a subject unsuitable to be treated with a therapeutic agent that inhibits cell migration is not responsive to treatment with the therapeutic agent. In some embodiments, the converting comprises increasing LPA levels above a predetermined threshold. In some embodiments, the converting comprises increasing LPA levels to the levels of a responder. In some embodiments, the inducing comprises increasing LPA levels above a predetermined threshold. In some embodiments, the inducing comprises increasing LPA levels to the levels of a responder.

In some embodiments, increasing LPA levels comprising administering an agent that increased LPA levels. In some embodiments, increasing LPA levels comprises increasing LPA protein levels. In some embodiments, increasing LPA levels comprises increasing LPA levels in a specific tissue. In some embodiments, increasing LPA levels comprises increasing LPA levels in the blood of the subject. In some embodiments, the blood is peripheral blood. In some embodiments, the blood is intestinal blood. In some embodiments, increasing LPA levels comprises decreasing LPA levels in the intestine. In some embodiments, increasing LPA levels comprises decreasing LPA levels in the gut. In some embodiments, LPA levels or activity are increased in blood and decreases in the intestine, gut or gut mucosa.

In some embodiments, increasing LPA levels comprises upregulating expression in the subject of at least one molecule that increases LPA levels. In some embodiments, the agent is a molecule that increases LPA levels. In some embodiments, the agent is a molecule that increases LPA function. In some embodiments, LPA levels are increased in blood. In some embodiments, LPA function is increased in blood. In some embodiments, increasing LPA levels comprises upregulation in the subject the activity of at least one molecule that increases LPA levels. In some embodiments, the molecule is an enzyme required for LPA biosynthesis. In some embodiments, the molecule stabilized LPA. In some embodiments, the molecule increases LPA's half-life. In some embodiments, the molecule increases transcription of a gene that encodes a protein that increases LPA levels. In some embodiments, the molecule increases translation of a mRNA that encodes a protein that increases LPA levels. In some embodiments, the molecule decreases LPA levels in the intestines. In some embodiments, the molecule blocks LPA entry or accumulation in the intestines. In some embodiments, the molecule blocks entry of LPA positive cells into the intestines. In some embodiments, the molecule blocks an LPA receptor in the intestines. In some embodiments, the molecule neutralizes LPA in the gut. In some embodiments, the molecule is LPA gut receptor antagonist. In some embodiments, the intestines is the gut mucosa.

In some embodiments, increasing LPA levels comprises downregulating expression in the subject of at least one molecule that decreases LPA levels. In some embodiments, increasing LPA levels comprises downregulation in the subject the activity of at least one molecule that decreases LPA levels. In some embodiments, the molecule is an enzyme required for LPA catabolism. In some embodiments, the molecule is an enzyme required for LPA breakdown or conversion to another molecule. In some embodiments, the molecule destabilized LPA. In some embodiments, the molecule decreases LPA's half-life. In some embodiments, the molecule decreases transcription of a gene that encodes a protein that decreases LPA levels. In some embodiments, the molecule decreases translation of a mRNA that encodes a protein that decreases LPA levels.

In some embodiments, the increasing comprises administering to the subject LPA. In some embodiments, the agent is LPA. In some embodiments, the LPA is bound to albumen. In some embodiments, the LPA is administered with albumen. In some embodiments, the increasing comprises administering to the subject an LPA precursor. In some embodiments, the agent is an LPA precursor. In some embodiments, the increasing comprises administering to the subject LPA and/or an LPA precursor. In some embodiments, the agent is LPA and/or an LPA precursor. In some embodiments, the LPA precursor is lysophosphatidylcholine (LPC). Any precursor, that can be converted to LPA in a subject may be administered. Any of these molecules may be administered with albumen or bound to albumen.

In some embodiments, upregulating expression of at least one molecule that increase LPA levels comprises administering a molecule that upregulated levels of ATX and/or CREB1 in the subject. In some embodiments, the agent is the molecule. In some embodiments, increasing activity of at least one molecule that increases LPA levels comprises administering an agonist of ATX, CREB1 or both. In some embodiments, the agent is the agonist. In some embodiments, increasing activity of at least one molecule that increases LPA levels comprises administering an activator of ATX, CREB1 or both. In some embodiments, the agent is the activator.

In some embodiments, downregulating expression of at least one molecule that decreases LPA levels comprises administering a molecule that downregulated levels of AGPAT3, SLC22A4, METTL9, and/or MBOAT2. In some embodiments, the agent is the molecule. In some embodiments, decreasing activity of at least one molecule that decreases LPA levels comprises administering an antagonist of AGPAT3, SLC22A4, METTL9 and/or MBOAT2. In some embodiments, the agent is the antagonist. In some embodiments, decreasing activity of at least one molecule that decreases LPA levels comprises administering an inhibitor of AGPAT3, SLC22A4, METTL9 and/or MBOAT2. In some embodiments, the agent is the inhibitor.

In some embodiments, increasing LPA activity comprises administering an LPA receptor agonist. In some embodiments, the agonist is a pan-LPA receptor agonist. In some embodiments, the agonist is a selective receptor agonist. In some embodiments, the agonist is a specific LPA receptor agonist. LPA receptors are well known in the art and include, but are not limited to, Lysophosphatidic acid receptor 1 (LPAR1), LPAR2, LPAR3, LPAR4, LPAR5 and LPAR6. In some embodiments, an LPA receptor agonist is an activating antibody. In some embodiments, the LPA receptor agonist is an LPA mimic. In some embodiments, the LPA receptor agonist is an LPA derivative. In some embodiments, the LPA receptor agonist is a small molecule. Any LPA receptor agonist known in the art may be employed. In some embodiments, increasing LPA activity in the blood comprises administering an LPA receptor antagonist to the intestines. In some embodiments, the LPA receptor antagonist is a blocking antibody. In some embodiments, the LPA receptor antagonist is a small molecule. In some embodiments, the LPA receptor antagonist is an LPA derivative. Any LPA receptor antagonist known in the art may be employed. Examples of LPA receptor agonists and antagonists can be found in Dong-Soon, Acta Pharma. Sinica, 2010, 31:1213-1222 for example. LPA receptor agonists include, but are not limited to NPSPA, NPTyrPA, N-acyl aminoethanol phosphoric acid (NAEPA), 1-oleoyl-2-O-methyl-rac-glycerophosphothionate (OMPT) LPA analogues, alkyl OMPT, 1-O-acyl-α-fluoromethylenephosphonate LPA analogues, dodecyl fatty alcohol phosphate, oleoyl-thiophosphate, methylene phosphonate LPA analogues, Farnesyl diphosphate, N-arachidonyl glycine, carba-cyclic phosphatidic acid, T-15, T-13 and α-hydroxymethylenephosphonate LPA analogues.

In some embodiments, the methods of the invention further comprise administering the therapeutic agent to the converted subject. In some embodiments, the methods of the invention further comprise administering the therapeutic agent the suitable subject. In some embodiments, the methods of the invention further comprise administering the therapeutic agent that blocks ITGA/B7 to the converted subject. In some embodiments, the methods of the invention further comprise administering the therapeutic agent that blocks ITGA/B7 to the suitable subject. In some embodiments, the methods of the invention further comprise administering the therapeutic agent that blocks ITGB7 to the converted subject. In some embodiments, the methods of the invention further comprise administering the therapeutic agent that blocks ITGB7 to the suitable subject. In some embodiments, the methods of the invention further comprise administering the therapeutic agent that blocks TNFα to the converted subject. In some embodiments, the methods of the invention further comprise administering the therapeutic agent that blocks TNFα to the suitable subject.

By another aspect, there is provided a method of reducing secretion of a pro-inflammatory cytokine from a cell, the method comprising contacting the cell with a therapeutic agent and an agent that increases LPA levels, function or both, thereby reducing secretion of a pro-inflammatory cytokine from a cell.

By another aspect, there is provided a pharmaceutical composition comprising a therapeutic agent and an agent that increases LPA levels, function or both.

By another aspect, there is provided a method of treating inflammation in a subject, the method comprising administering to the subject a therapeutic agent and an agent that increases LPA levels, function or both.

In some embodiments, the method of reducing secretion comprises administering LPA, an LPA precursor, or both. In some embodiments, the method of reducing secretion comprises administering LPA. In some embodiments, the therapeutic agent is an agent that inhibits cell migration. In some embodiments, the agent is an anti-integrin blocking antibody. In some embodiments, the agent is not an anti-pro-inflammatory cytokine antibody.

In some embodiments, the cell is in vitro. In some embodiments, the cell is in vivo. In some embodiments, the cell is ex vivo. In some embodiments, the cell is in blood. In some embodiments, the cell is in a local site of inflammation. In some embodiments, the local site of inflammation is a mucosa. In some embodiments, the local site of inflammation is the gut.

In some embodiments, the method of treating comprises administering the pharmaceutical composition of the invention. In some embodiments, the therapeutic agent is administered, before, after or concomitantly with the agent that increases LPA levels, function or both. In some embodiments, the agents are administered in separate pharmaceutical compositions.

In some embodiments, the pharmaceutical composition comprises LPA, a precursor of LPA or both and therapeutic agent. In some embodiments, the pharmaceutical composition comprises an anti-integrin antibody and an agent that increases LPA levels, function or both. In some embodiments, the anti-integrin antibody is an anti-ITGA4/B7 antibody. In some embodiments, the pharmaceutical composition comprises an agent that inhibits cell migration and an agent that increases LPA levels, function or both. In some embodiments, the pharmaceutical composition does not comprise an anti-pro-inflammatory cytokine antibody.

In some embodiments, the pharmaceutical composition comprises a pharmaceutically acceptable carrier, excipient or adjuvant. Each possibility represents a separate embodiment of the invention. In some embodiments, the pharmaceutical composition is formulated for systemic administration. In some embodiments, the pharmaceutical composition is formulated for local administration. In some embodiments, local administration is to a location of inflammation. In some embodiments, local administration is to a mucosa. In some embodiments, local administration is to the gut.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

According to another aspect, there is provided a kit comprising at least two detection molecules selected from: a detection molecule specific to ATX, a detection molecule specific to CREB1, a detection molecule specific to AGPAT3, a detection molecule specific to SLC22A4, a detection molecule specific for METTL9 and a detection molecule specific to MBOAT2. In some embodiments, the kit comprises 1, 2, 3, 4, 5, or 6 of the detection molecules. Each possibility represents a separate embodiment of the invention. In some embodiments, the kit consists of the detection molecule.

According to another aspect, there is provided a kit comprising a therapeutic agent and an agent that increases LPA levels, function or both.

In some embodiments, the kit consists of a therapeutic agent and an agent that increases LPA levels, function or both. In some embodiments, the kit comprises a tag or label stating that the contents of the kit are to be used together. In some embodiments, the therapeutic agent comprises a tag stating it is to be used with an agent that increases LPA levels, function or both. In some embodiments, the agent that increases LPA levels, function or both comprises a tag stating it is to be used with the therapeutic agent.

In some embodiments, the kit is for use in performing a method of the invention. In some embodiments, the kit is for treatment. In some embodiments, the kit is for diagnostics. In some embodiments, the kit is for diagnostics and treatment.

In some embodiments, the kit comprises no more than 4, 5, 6, 7, 8, 9, 10, 15, 20, 25, 50, 75, 100, 200, 300, 500 or 100 detection molecules. Each possibility represents a separate embodiment of the invention. In some embodiments, the detection molecule detects protein. In some embodiments, the detection molecule detects RNA. In some embodiments, the detection molecule is an antibody. In some embodiments, the detection molecule is a hybridization probe. In some embodiments, the detection molecule is a pair of primers. In some embodiments, the primers are PCR primers. In some embodiments, the detection molecule is a nucleic acid sequence complementary to an mRNA that encodes the target protein. In some embodiments, the detection molecules are connected to a solid scaffold. In some embodiments, the scaffold is inorganic.

In some embodiments, the kit further comprises a detection molecule specific to LPA. In some embodiments, the kit further comprises at least one molecule for administration to a subject unsuitable for treatment with a therapeutic agent to convert the subject to a suitable subject. In some embodiments, the kit further comprises a therapeutic agent.

In some embodiments, a kit of the invention is for determining the suitability of a subject in need thereof to be treated with a therapeutic agent. In some embodiments, the kit is for converting an unsuitable subject to a suitable subject. In some embodiments, a kit of the invention is for determining suitability for treatment. In some embodiments, the treatment is treatment of IBD. In some embodiments, the treatment is treatment for inflammation.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Materials and Methods

### Sample Collection and Patient Clinical Characterization

The exploratory study cohort consisted of 73 whole blood samples from 22 IBD (UC and CD) patients, who received Vedolizumab treatment at the Rambam Health Care Campus (RHCC) and met the study inclusion criteria. Patients that had past exposure to Vedolizumab, or patients who had active infection including febrile diseases or intra-abdominal or perianal abscess were excluded.

Samples were collected at the following time-points: pretreatment, 2 weeks and 14 weeks post first treatment and following one year of treatment for those patients who received long-term care. Patients were classified for response by clinical score after one-year follow-up and were clinically characterized as detailed in table 2.

TABLE 2
Disease characteristics of patients and clinical outcomes from cohort 1

| | | Non- | |
|---|---|---|---|
| | Responders | responders | |
| | (n = 12) | (n = 10) | P** |
| Disease (CD/UC) | 7/5 | 5/5 | 0.70 |
| | (58.3%/ 41.7%) | (50%/ 50%) | |
| Age (years)* | 38.1 ± 22.9 | 41.2 ± 21.7 | 0.47 |
| Gender (F/M) | 7/5 | 4/6 | 0.40 |
| | (58.3%/ 41.7%) | (40%/ 60%) | |
| clinical 2 w | 1/7/0 | 5/5/0 | 0.09 |
| (0/1/2) *** | (12.5%/ 87.5%/0%) | (50%/ 50%/ 0%) | |
| clinical 14 w | 1/5/2 | 7/2/1 | 0.05 |
| (0/1/2) *** | (12.5%/62.5%/25%) | (70%/20%/10%) | |
| clinical 26 w | 0/4/5 | 8/2/0 | 0.01 |
| (0/1/2) *** | (44.5%/55.5%) | (80%/20%/0%) | |
| clinical 52 w | 0/0/12 | 10/0/0 | <.001 |
| (0/1/2) *** | (0%/0%/100%) | (100%/0%/0%) | |
| CRP pretreatment | 16.7 ± 6.9 | 11.6 ± 3.9 | 0.99 |
| (max normal | | | |
| value 5) | | | |
| CRP 2 w | 12.8 ± 6.1 | 6.5 ± 1.4 | 0.86 |
| (max normal | | | |
| value 5) | | | |
| CRP 14 w | 6.4 ± 1.6 | 5.8 ± 1.1 | 1.00 |
| (max normal | | | |
| value 5) | | | |
| Albumin | 3.7 ± 0.1 | 3.1 ± 0.1 | 0.01 |
| pretreatment | | | |
| Albumin 2 w | 3.5 ± 0.2 | 3.2 ± 0.1 | 0.08 |
| Albumin 14 w | 3.9 ± 0.2 | 3.5 ± 0.2 | 0.10 |

*Expressed by mean ± SEM
**Chi squared test for categorical data; Wilcoxon test for continuous measure
*** Primary clinical response to Vedolizumab, defined as clinical improvement according to treating physician: 0—None; 1—partial; 2—full

A second validation cohort was also analyzed, containing 70 whole blood samples from 34 IBD (UC and CD) patients, who received Vedolizumab treatment at the Rambam Health Care Campus (RHCC) and met the study inclusion/exclusion criteria. Two healthy controls were also included. Patients were classified for response by clinical scoring after 14-26 weeks of follow-up and their clinical characteristics are depicted in Table 3.

TABLE 3
Disease characteristics of patients and clinical outcomes from cohort 2

| | Responders | Non-responders | |
|---|---|---|---|
| | (n = 18) | (n = 16) | P** |
| Disease (CD/UC) | 10/8 | 8/8 | 0.74598 |
| | (55.6%/44.4%) | (50%/50%) | |
| Age (years)* | 45.7 ± 4.6 | 44.3 ± 6.2 | 0.879063 |
| Gender (F/M) | 13/5 | 6/10 | 0.041824 |
| | (72.2%/27.8%) | (37.5%/62.5%) | |
| CRP pretreatment | 10.3 ± 2.7 | 24.9 ± 6.2 | 0.035621 |
| (max normal value 5)* | | | |
| CRP 2 w | 9.9 ± 3.0 | 24.5 ± 6.8 | 0.028232 |
| (max normal value 5)* | | | |
| CRP 14 w | 7.6 ± 3.2 | 22.2 ± 7.8 | 0.014011 |
| (max normal value 5)* | | | |
| Albumin | 4.0 ± 0.5 | 3.6 ± 0.1 | 0.008445 |
| pretreatment* | | | |
| Albumin 2 w* | 3.7 ± 0.6 | 3.6 ± 0.1 | 0.051331 |
| Albumin 14 w* | 4.1 ± 0.6 | 3.7 ± 0.1 | 0.005208 |
| Past therapy- | 5/13 | 5/11 | 0.824479 |
| Thiopurines (N/Y) | (27.8%/72.2%) | (31.3%/68.7%) | |
| Past therapy - | 11/7 | 6/10 | 0.169327 |
| Anti-TNF (N/Y) | (61.1%/38.9%) | (37.5%/62.5%) | |
| Past therapy - | 15/3 | 14/2 | 0.732046 |
| MTX (N/Y) | (83.3%/16.7%) | (87.5%/12.5%) | |
| Initiation co-therapy- | 11/7 | 9/7 | 0.773753 |
| Steroids (N/Y) | (61.1%/38.9%) | (56.3%/43.7%) | |
| Initiation co-therapy- | 12/6 | 12/4 | 0.594525 |
| Thiopurines (N/Y) | (66.7%/33.3%) | (75%/25%) | |
| Week 2 co-therapy | 12/6 | 9/7 | 0.532723 |
| Steroids (N/Y) | (66.7%/33.3%) | (56.3%/43.7%) | |
| Week 2 co-therapy | 12/6 | 12/4 | 0.594525 |
| Thiopurines | (66.7%/33.3%) | (75%/25%) | |
| Week 14 co-therapy | 14/4 | 9/7 | 0.180476 |
| Steroids (N/Y) | (77.8%/22.2%) | (56.3%/43.7%) | |
| Week 14 co-therapy | 12/6 | 12/4 | 0.594525 |
| Thiopurines (N/Y) | (66.7%/33.3%) | (75%/25%) | |

Upon collection, all samples were pre-processed and properly maintained for downstream analysis of mRNA expression by gene array.

### Peripheral Blood Gene Expression Analysis

### Gene Expression Data Preprocessing

Whole blood was maintained in PaxGene tubes. RNA was extracted and assayed using Affymetrix Clariom S chips. The raw gene array data were processed to obtain a log 2 expression value for each gene probe set using RMA (robust multichip average) method available in affymetrix v1.50.0 R package. Probe set annotation was performed using affycoretools and clariomshumantranscriptcluster.db packages in R. Data was further adjusted for batch effect using empirical Bayes framework applied by the Combat R package.

### Estimated Cell Proportion Scores Based on Gene Expression Data

xCell, which is a computational deconvolution method based on ssGSEA enrichment of cell specific signatures for estimation of abundance scores of immune cell types from the gene expression data, was used. Only cells that had non-zero values in at least 75% of the samples were included.

Based on the estimated cell proportions, the gene expression data was adjusted for variation across samples in the major cell type proportions including CD4, CD8, CD19, CD14, NK and granulocytes, using linear regression by calculated residual values (CellMix R package).

### Integrin Downstream Signaling Targeted Analysis for Response Dynamic Characterization and Baseline-Based Response Prediction

Genes involved in integrin downstream signaling were mapped using Metacore (Thomson Reuters system biology solution) and based on literature (Giancotti and Ruoslahti, 1999; Sun et al., 2014). Overall 80 integrin related genes were included in the initial analysis. Changes in the mapped integrin-related genes in responders over time (0 w-14 w) were identified using paired t test. This was followed by PCA (principle component analysis) as a dimensionality reduction tool that enables representation of the integrin signaling-related variance observed in samples by two major components. Ultimate responders in terms of integrin related response were ordered based on the PCA based—Euclidian distance.

To detect integrin downstream concomitant changes, genes that were highly correlated with the PC1 of the integrin related PCA in responders were searched for. Genes that presented absolute Spearman's rank correlation coefficient above 0.75, and were differentially expressed (limma R package, patient code was defined as random effect) between visits in responders were included.

Based on these results, which characterize normal drug response dynamics, next, differentially expressed genes between responders and non-responders 14 weeks post first treatment were identified. Network propagation was used to enhance the biological signal by adding known interacting protein genes using ConsensusPathDB.

At the final stage of the analysis, differential expression of the above genes was tested, between responders and non-responders pre-treatment using Wilcoxon test.

Genes that were differentially expressed and presented coherent biological pathway relatedness were tested for prediction at baseline. The combined predictive value of these genes was examined using logistic regression. For prediction performance evaluation a receiver operating characteristic curve (AUC) was constructed. For internal validation, 10-fold cross-validation was performed to control for overfitting (glm and cvAUC R packages).

### Measurement of Serum LPA Levels by ELISA (Enzyme-Linked Immunosorbent Assay)

Serum LPA levels from 30 patients (14 and 16 responding and non-responding patients correspondingly) was quantified by ELISA using ELISA Kit (Cloud Clone Corp.) according to manufacturer's instructions. Concentrations were calculated by comparing the sample absorbance to standard curves.

### Example 2: Responding Patients Show Increased Estimated Proportions of CD4 T Cell Subsets by Deconvolution

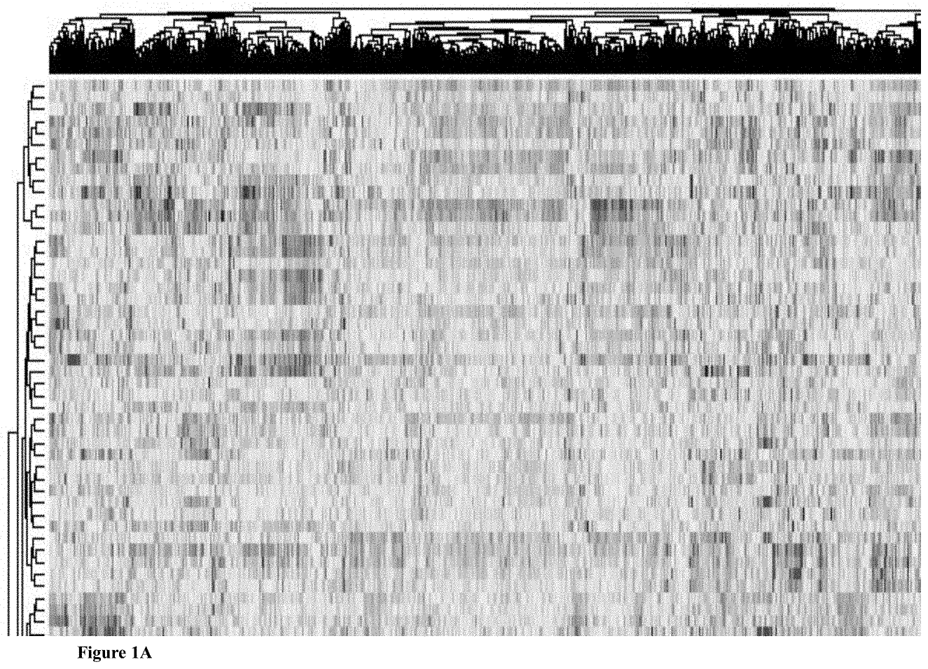

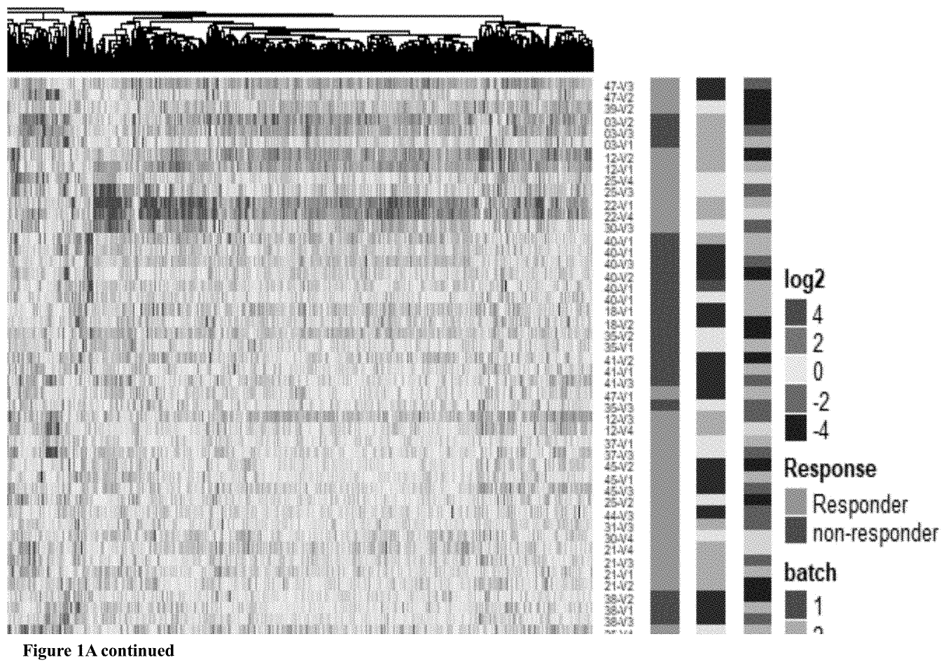

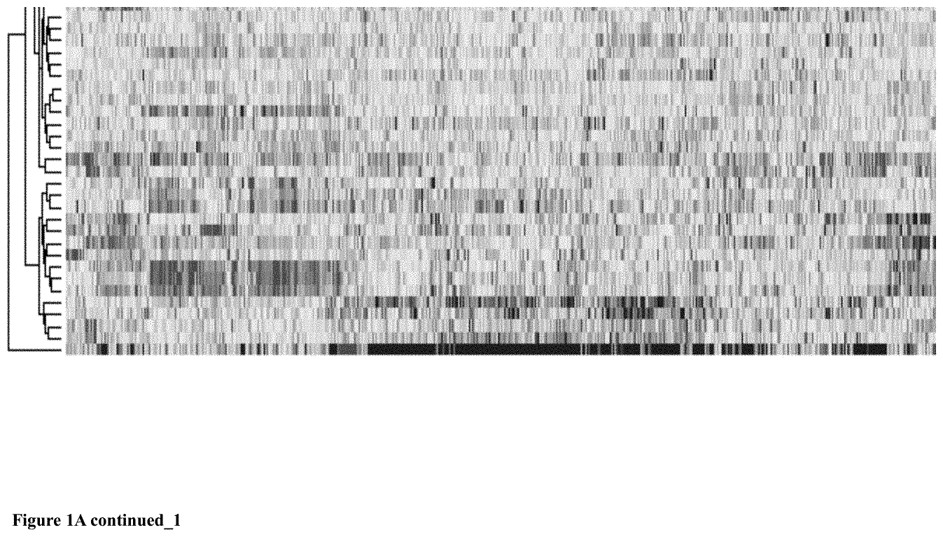

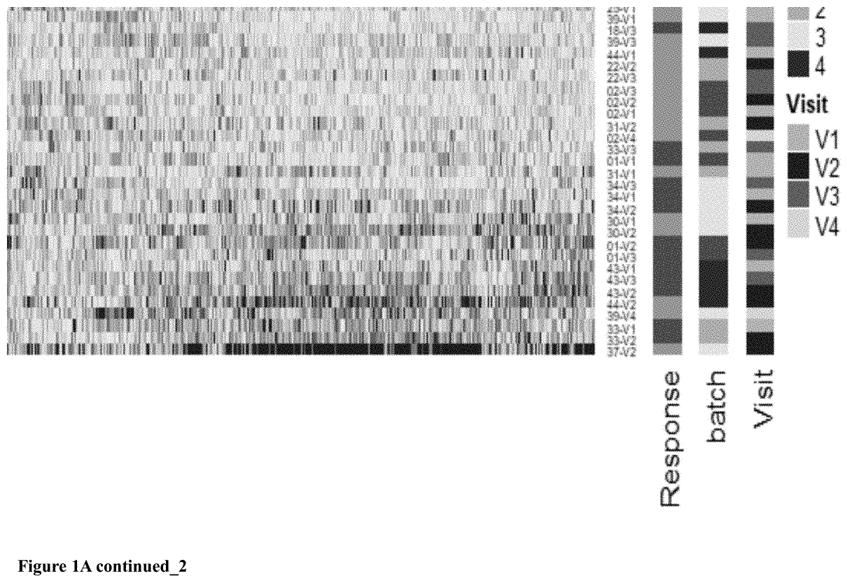

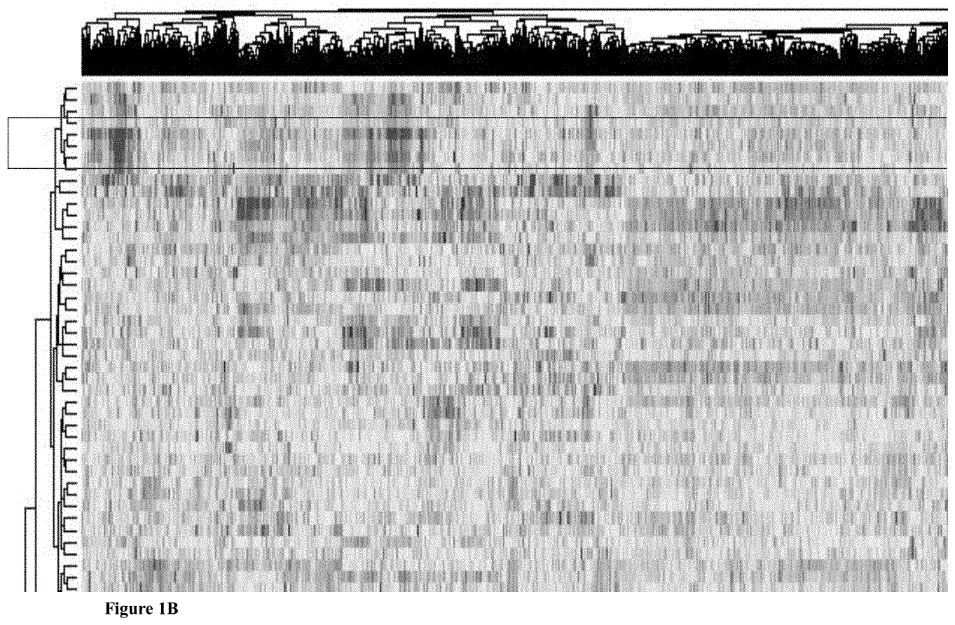

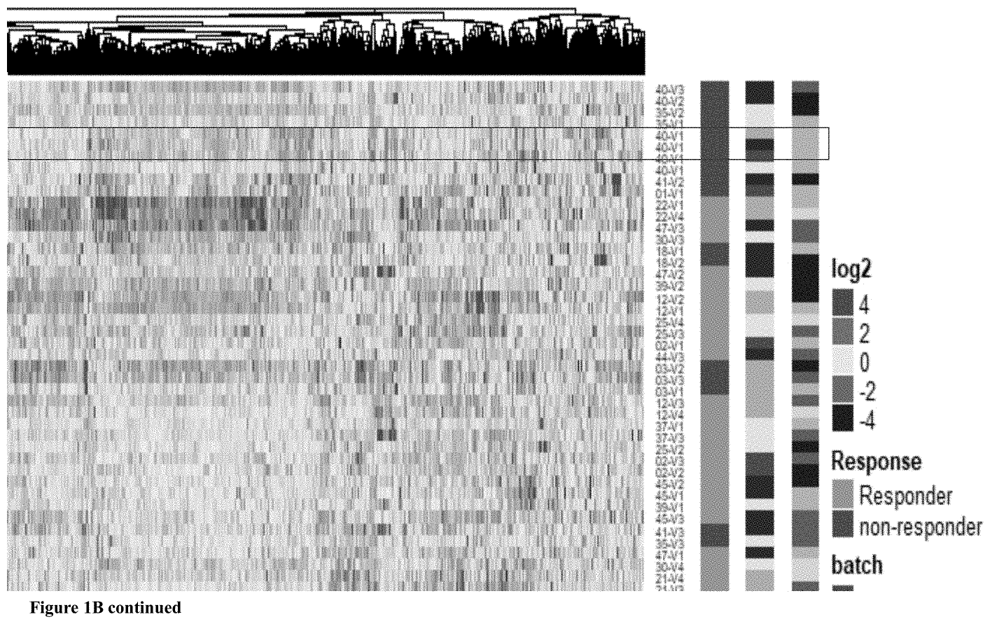

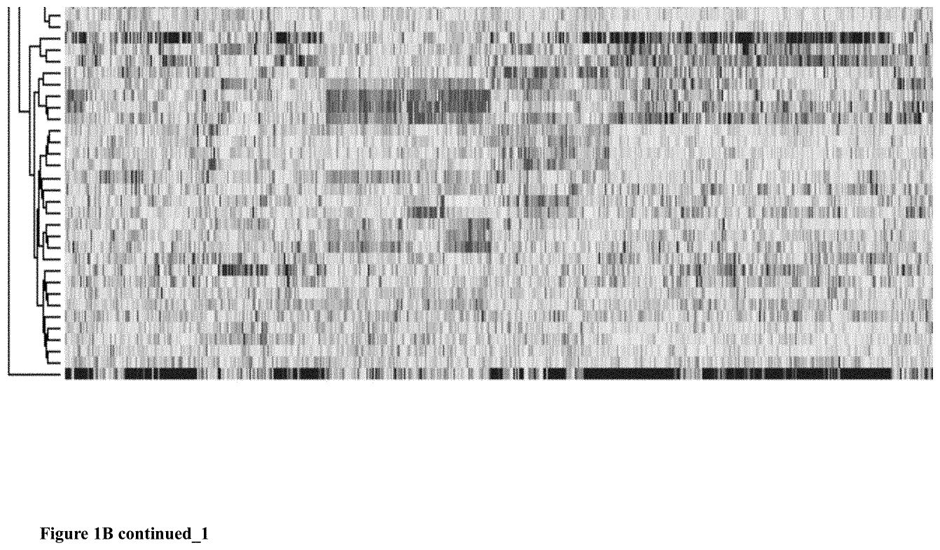

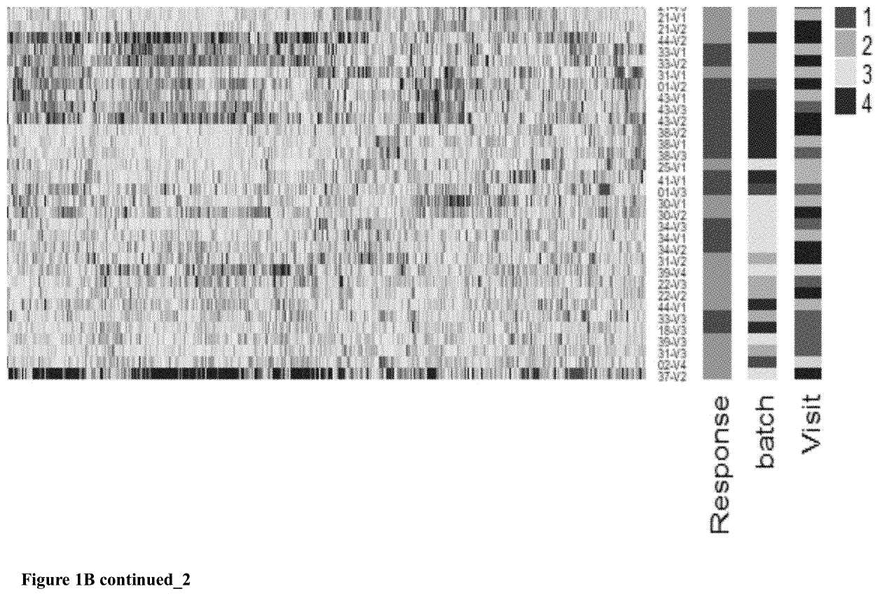

Successful batch correction was confirmed by close clustering post batch correction, of a common sample that was assayed in all batches (40-V1) (FIG. 1A-B).

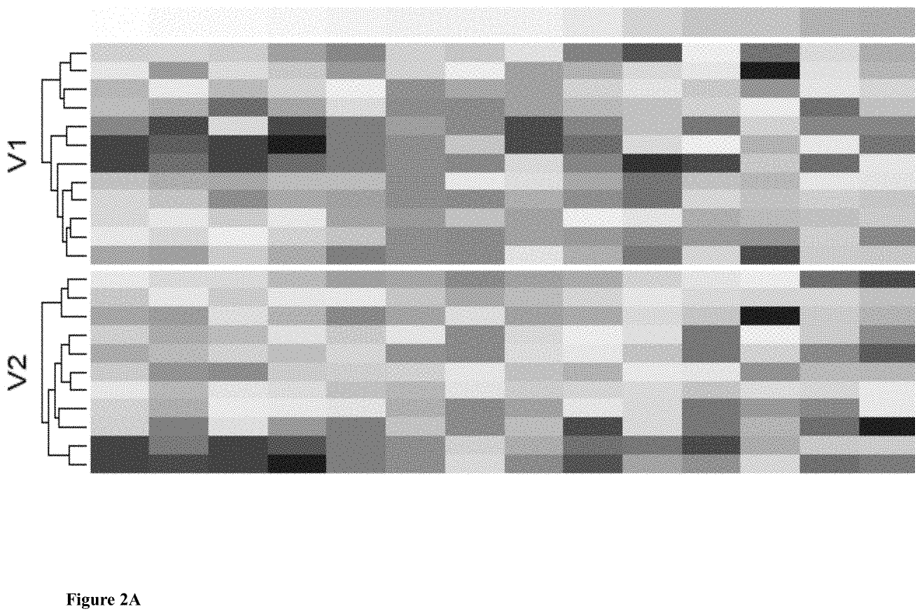

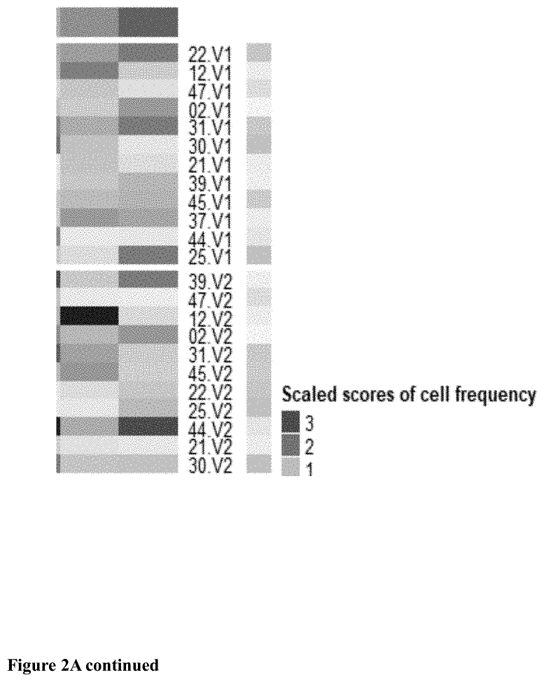

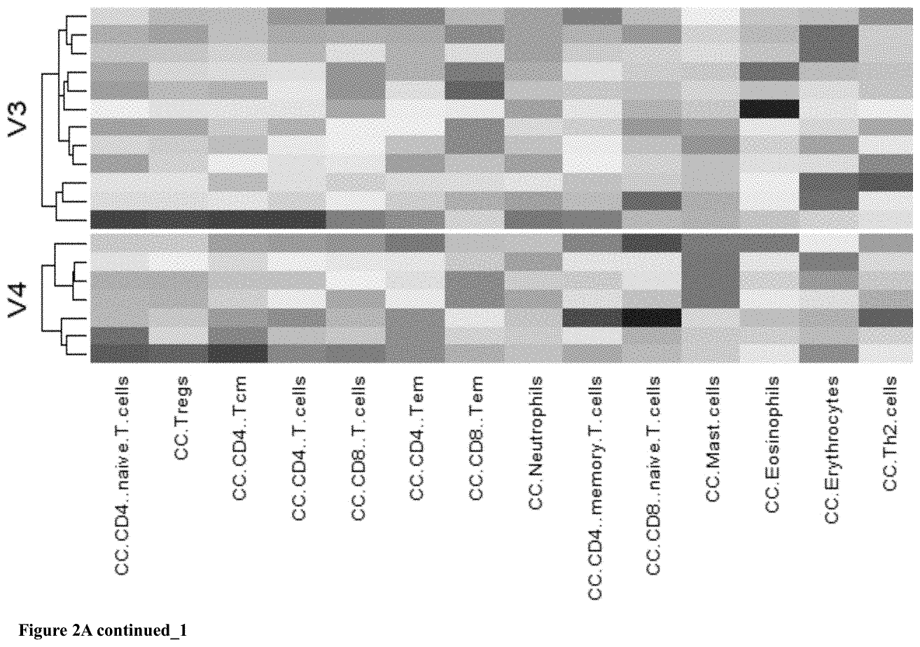

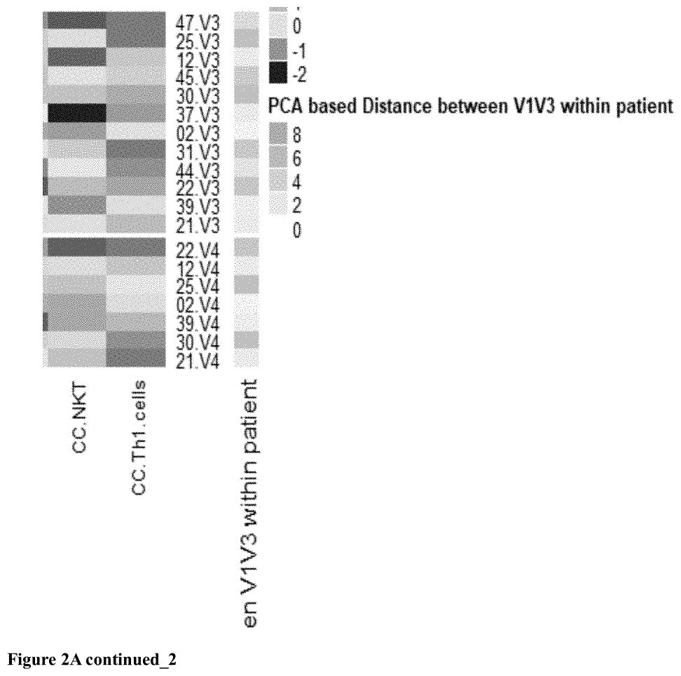

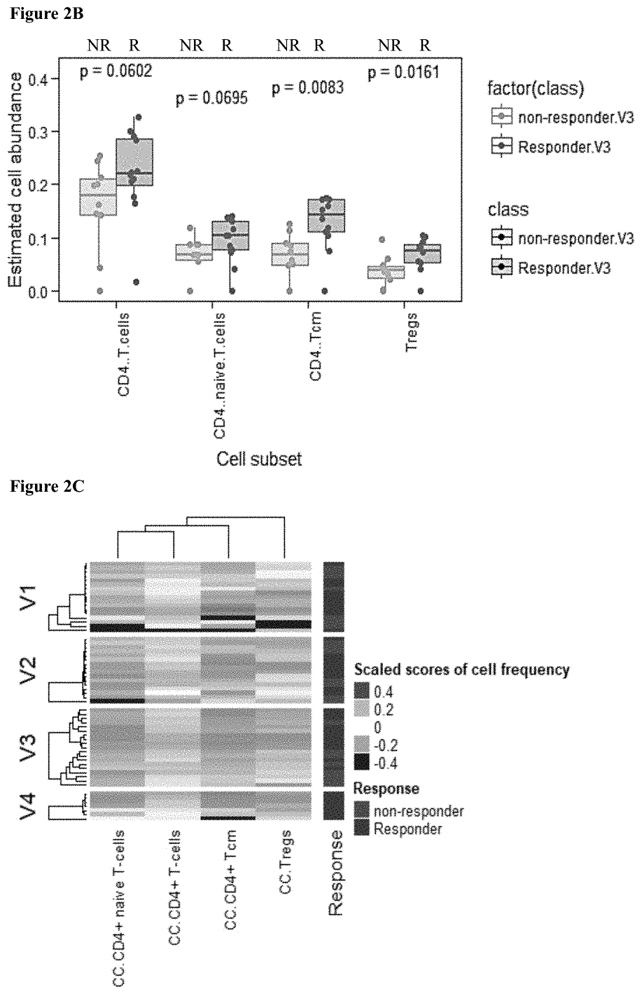

Based on the cell centered deconvolution analysis results, it was found that the most significant change in cell abundance in responding patients between baseline and 14 weeks post first treatment was observed in CD4 T cell population in general, attributed particularly to changes in naïve CD4, CD4 Tcm (central memory) and regulatory T cell subsets (FIG. 2A). These subsets were also differentially abundant in visit 3 (V3, 14 w post treatment) between responders and non-responders (FIG. 2B), presenting higher estimated frequency score in responders (p<0.1, Wilcoxon test). The dynamics of these cells in all visits in responders and non-responders is shown in FIG. 2C.

The increased deconvolved estimated proportions in CD4 subsets corroborate previous studies that reported an increased level of total CD4 counts and increased Treg proportions in peripheral blood.

### Example 3: Responding Patients have Reduced Estimated Proportion Score of Tregs in Intestinal Tissue while Non-Responders do not Show Abundance Change

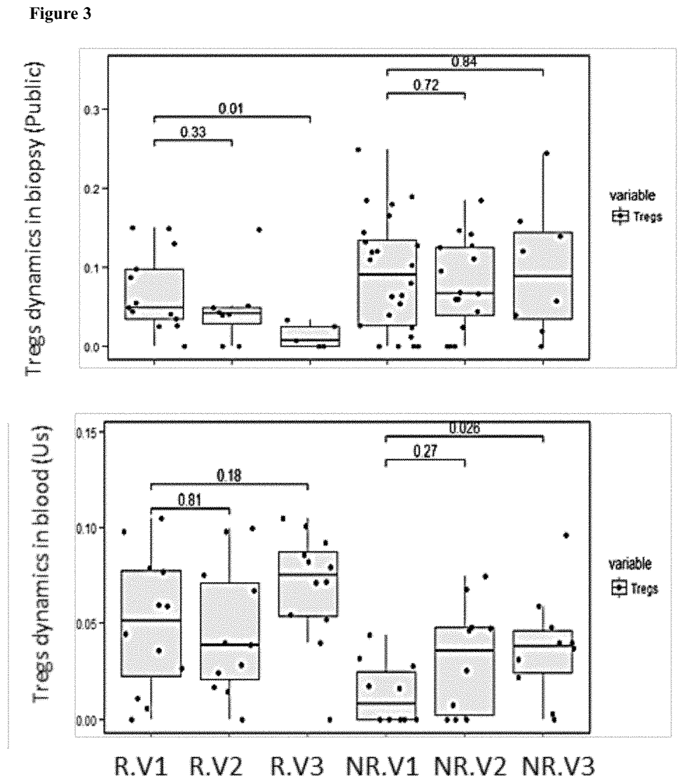

Estimated cell proportions in intestinal tissue were tested using a publicly available dataset (GEO73661). The most significant change in both peripheral blood and intestinal tissue was observed in Tregs (FIG. 3). In tissue, Vedolizumab reduces Tregs frequency, while non-responders are unaffected. Although Treg accumulation is observed in blood of non-responders, tissue Tregs are not reduced.

### Example 4: Accounting for Cells, Unmasks Differential Regulation

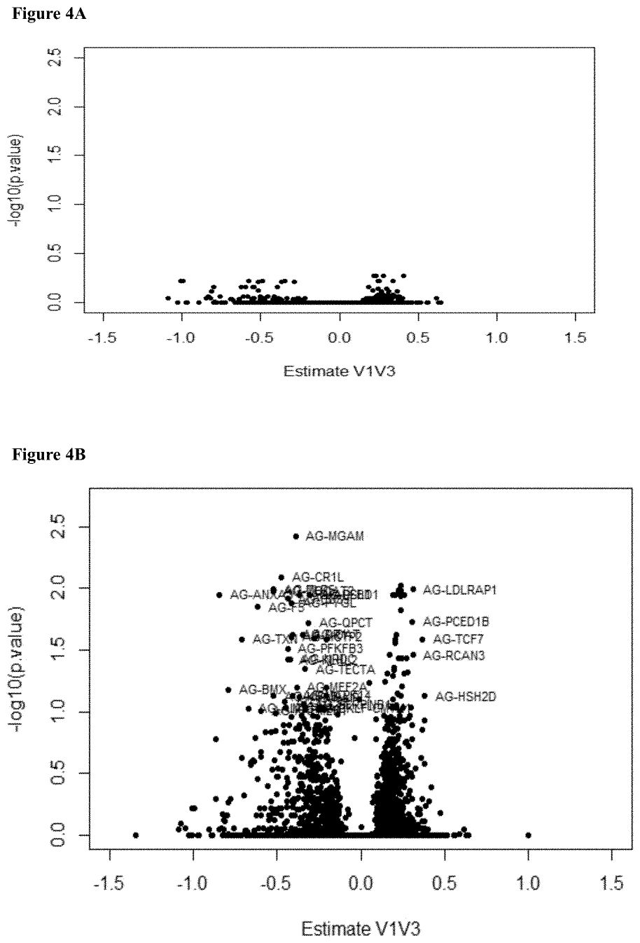

By standard gene differential expression analysis using linear mixed-effects model (defining patient code as a random effect), only a few changes in gene expression between visits in responding patients were detected. Based on these results, and the variability in estimated cell proportions, the signal observed in responders dynamics was enhanced via a cell-centered approach that relies on adjusting the expression data for variation across samples in the major cell type proportions (CD4, CD8, CD19, CD14, NK and granulocytes). This analysis allows to detect differential regulation that does not stem from cell proportion differences, which may be masked in standard analysis (FIG. 4A-B).

### Example 5: Responders and Non-Responders have Similar Response to the Drug Target—Integrin a4/b7 Genes in Blood

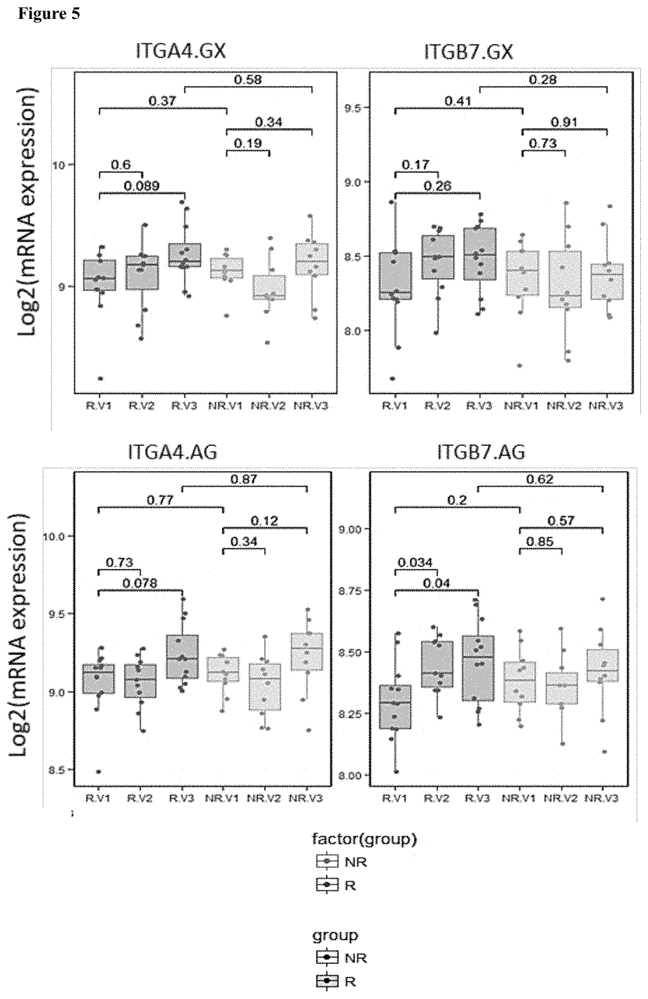

As Vedolizumab is a monoclonal antibody which targets α4β7 integrin, the drug target expression (pre and post adjustment to major cell type proportions) was first tested in responding and non-responding patients, pre-treatment and 2 weeks and 14 weeks post first treatment (FIG. 5). As shown, responders showed an increase in β7 and α4 adjusted expression post treatment in peripheral blood. Non-responders presented a trend of increased expression, but without significance due to high variability between patients. No significant differences were detected between responders and non-responders within visits.

Although responding and non-responding patients exhibited similar expression of the drug target, it was hypothesized that response differences between groups may be related to downstream integrin signaling through effector proteins, rather than to the target itself.

### Example 6: Responders Present Changes in Integrin Downstream Signaling Following Therapy and Integrin-Associated Dynamics

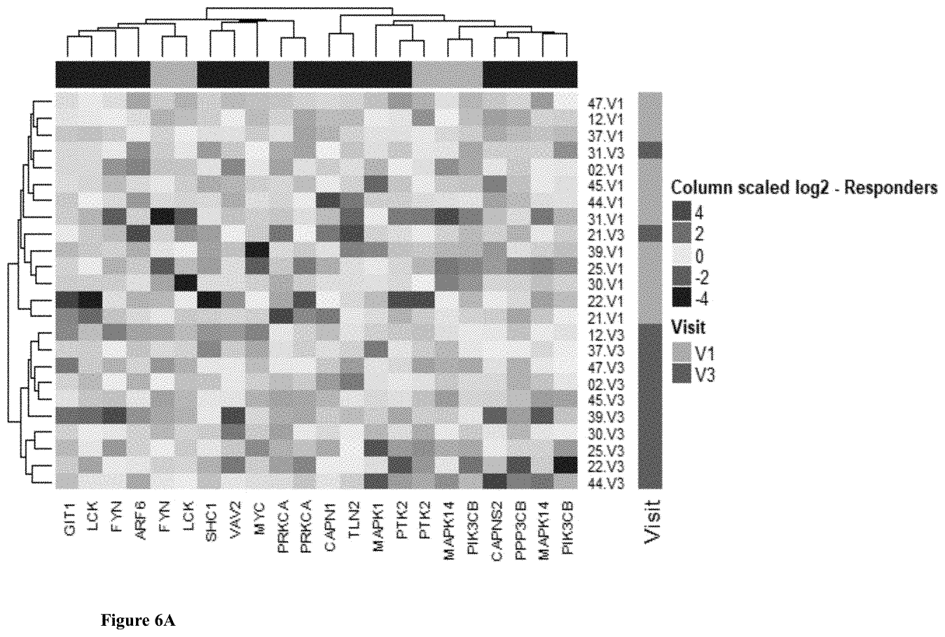

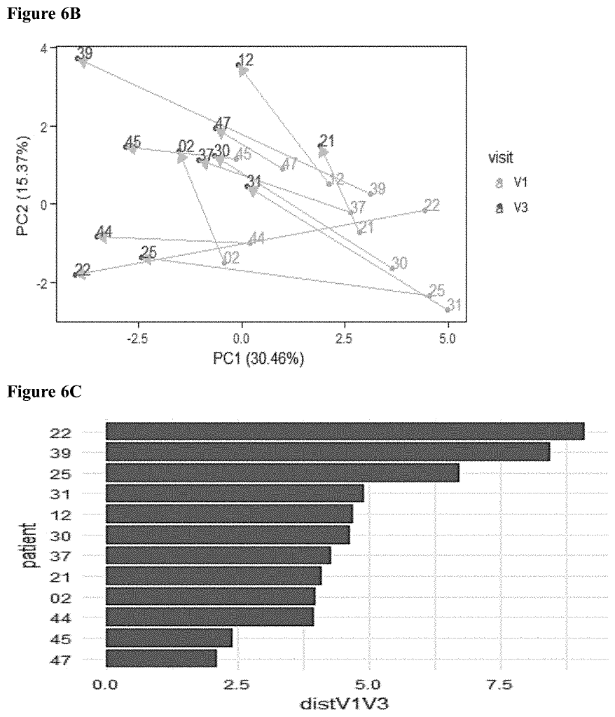

Targeted analysis revealed changes in the integrin related genes in responders over time (0 w-14 w) using paired t test (p<0.05). (FIG. 6A). Using PCA, there was observed a clear separation of the samples by visits on the first axis (FIG. 6B). By calculating the distance between baseline and 14 weeks post first treatment within each patient, ultimate responders could be identified in terms of integrin related response, i.e. patients that exhibited a larger distance between 14 w and 0 w, presented more significant change in integrin signaling, and as a responder, the change was likely to be clinically beneficial (FIG. 6C).

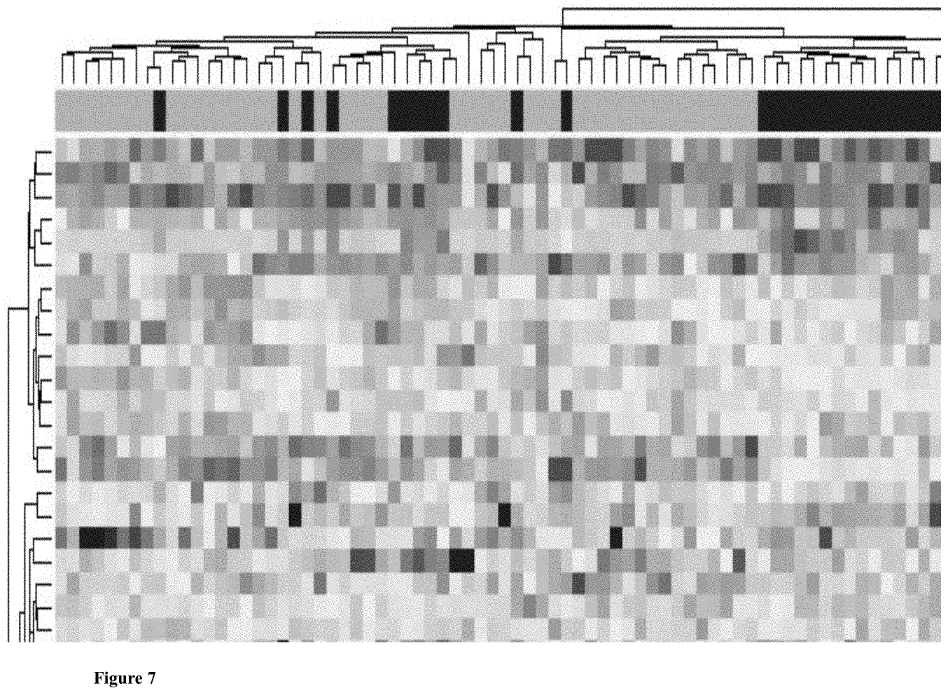

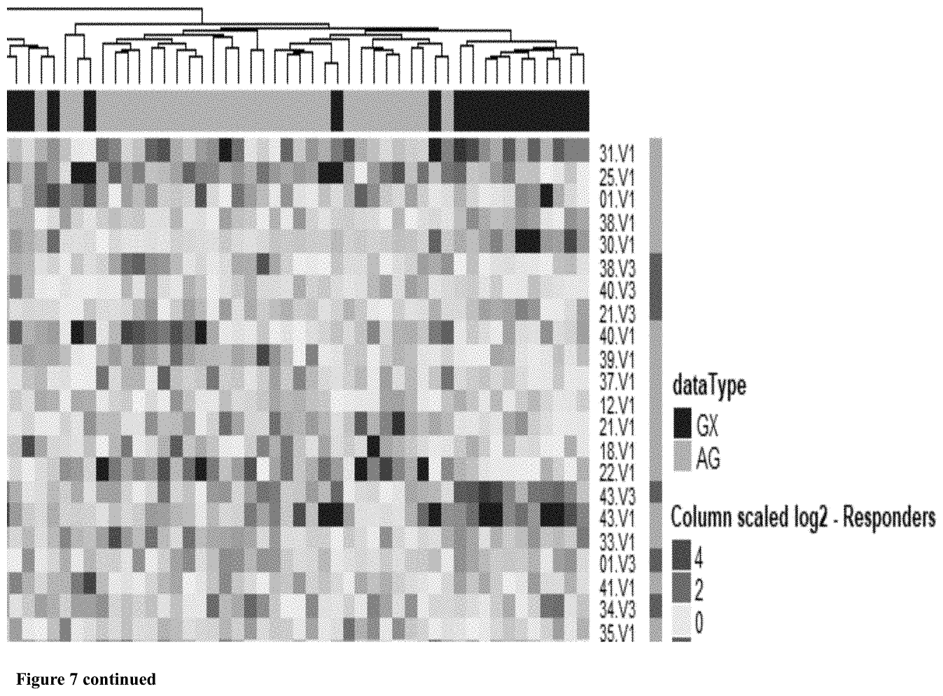

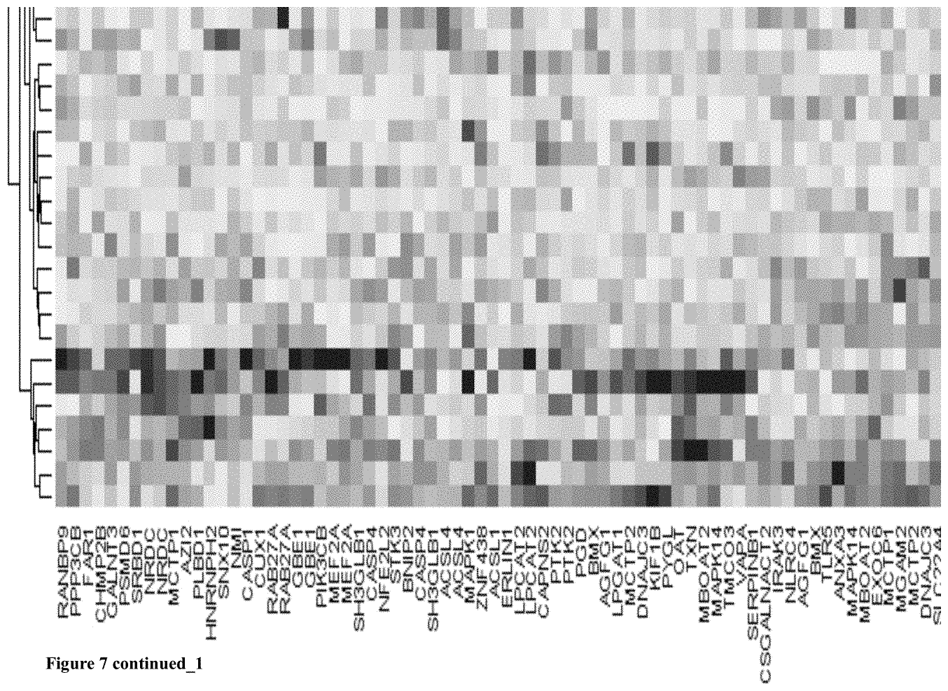

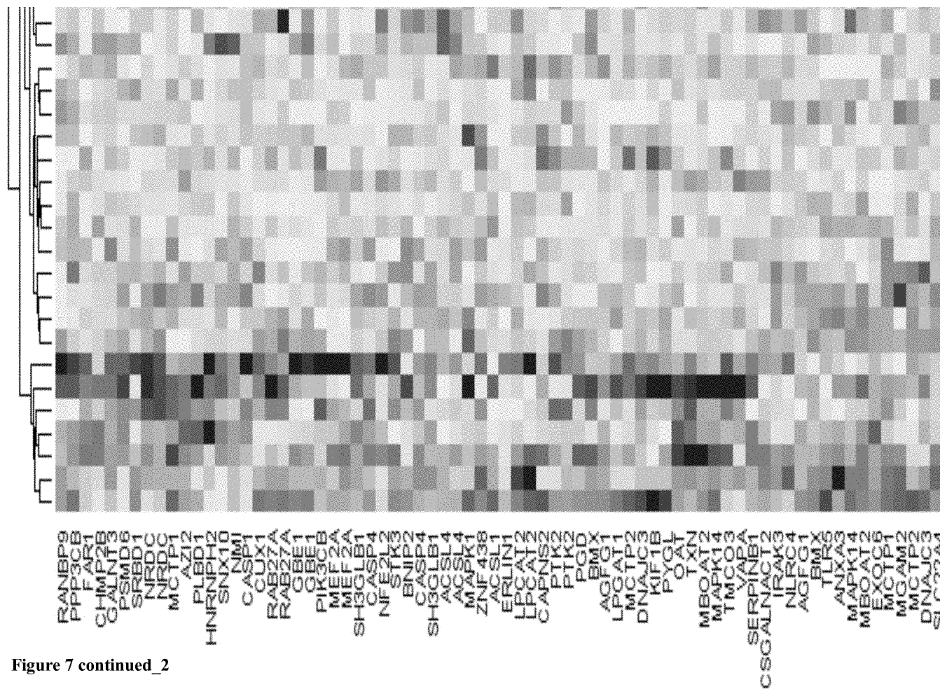

Using correlation-based analysis with the first PC of the integrin signaling-related PCA, additional related gene expression changes were identified that are associated with the integrin downstream signaling, as shown in FIG. 7. These alternations were defined as a normal integrin associated response for Vedolizumab treatment.

### Example 7: Responding Patients Present Increased Pre-Treatment LPA Synthesis Capacity in the Blood

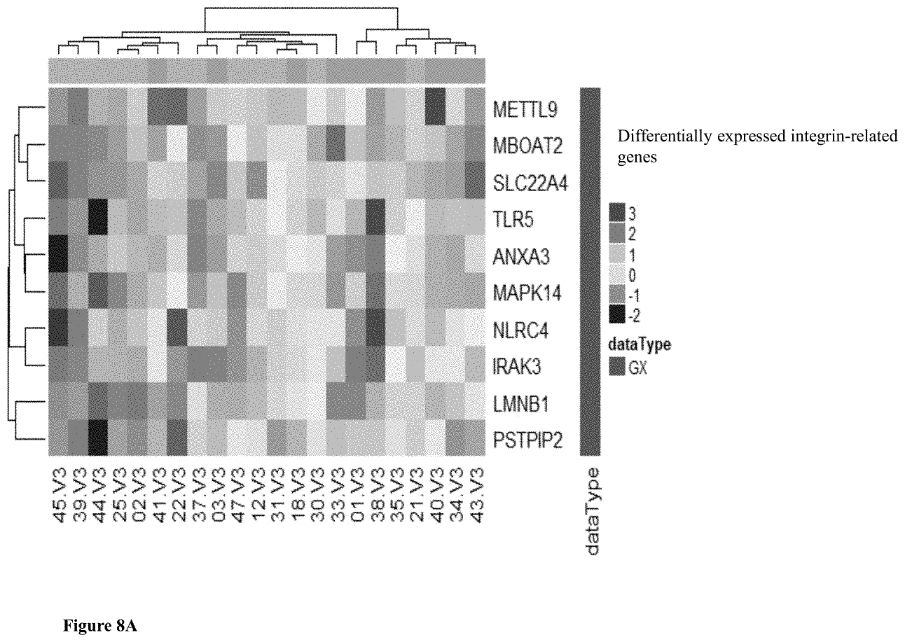

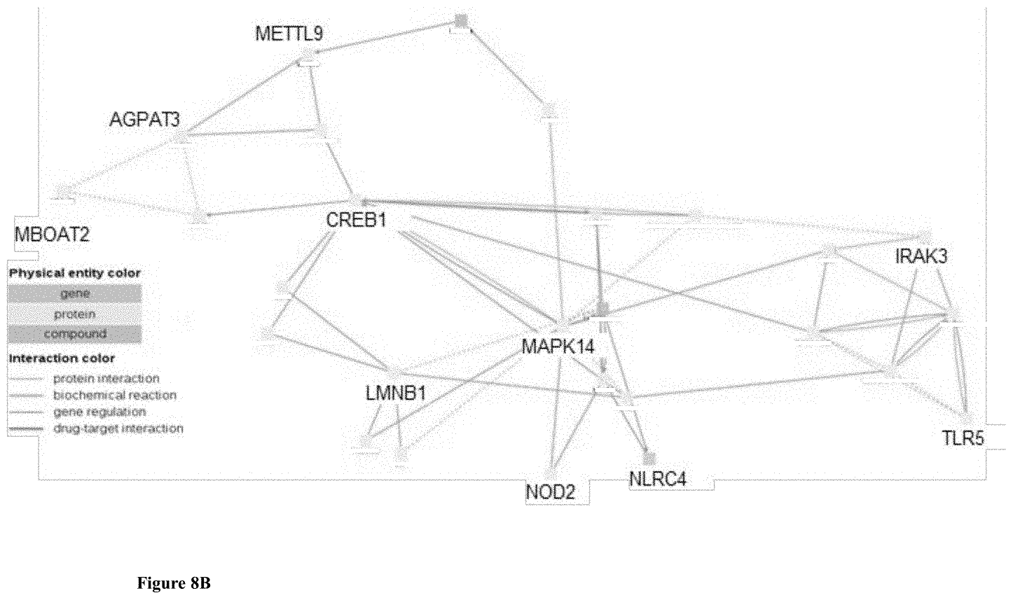

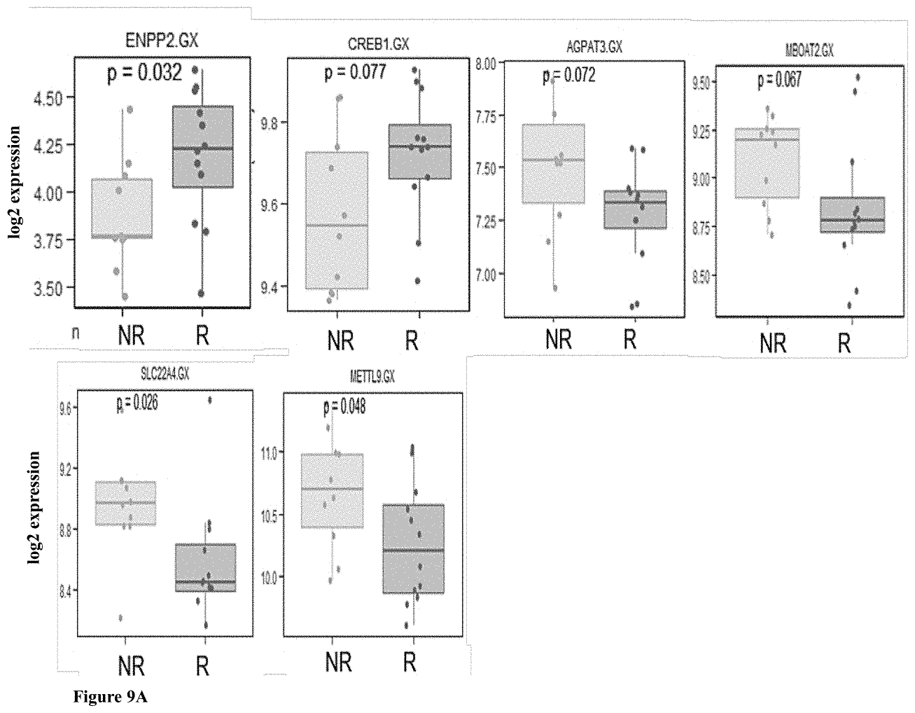

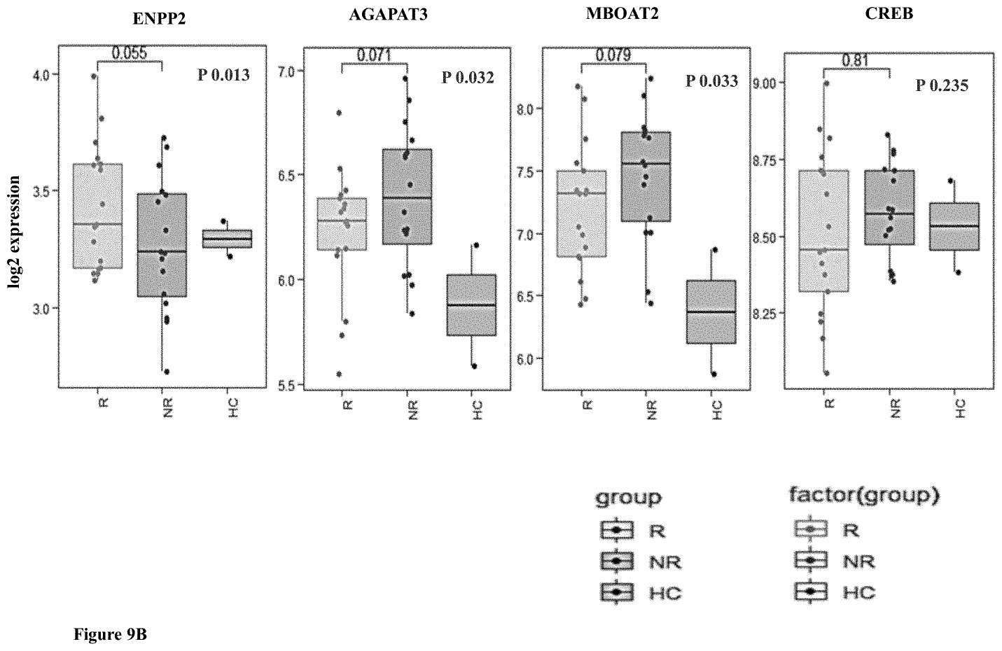

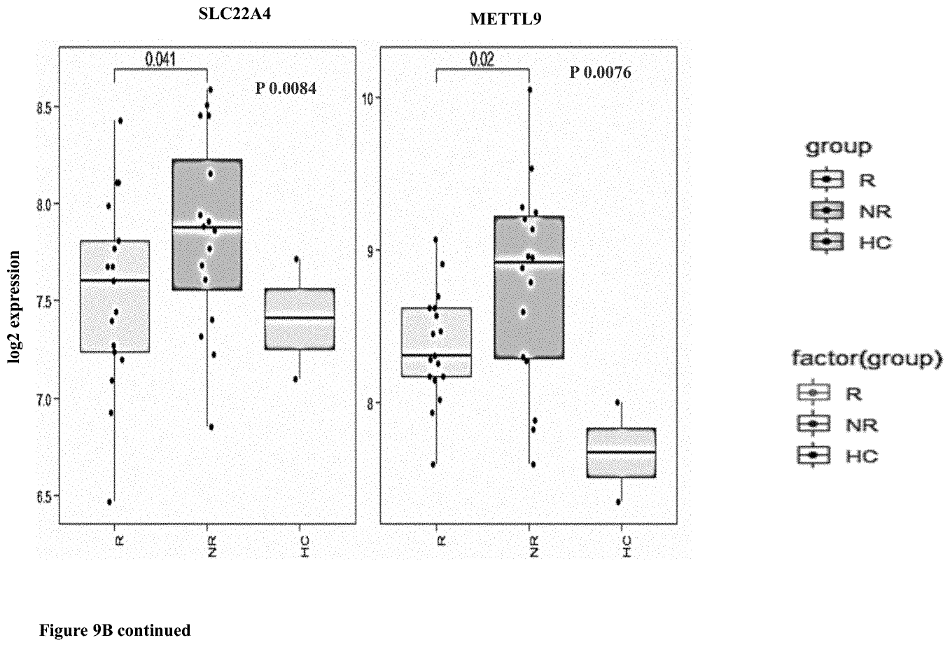

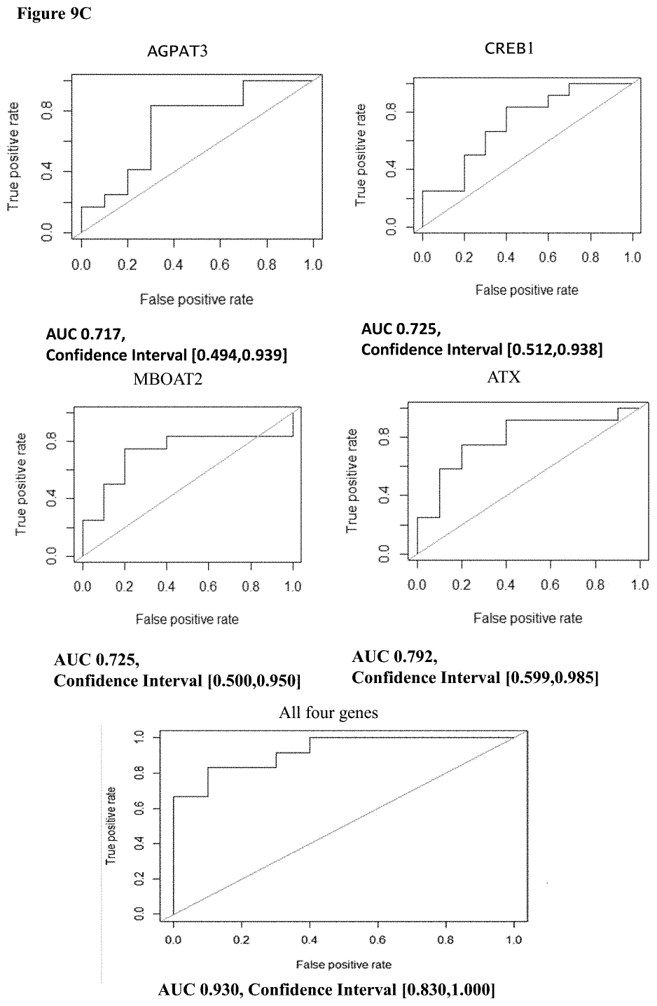

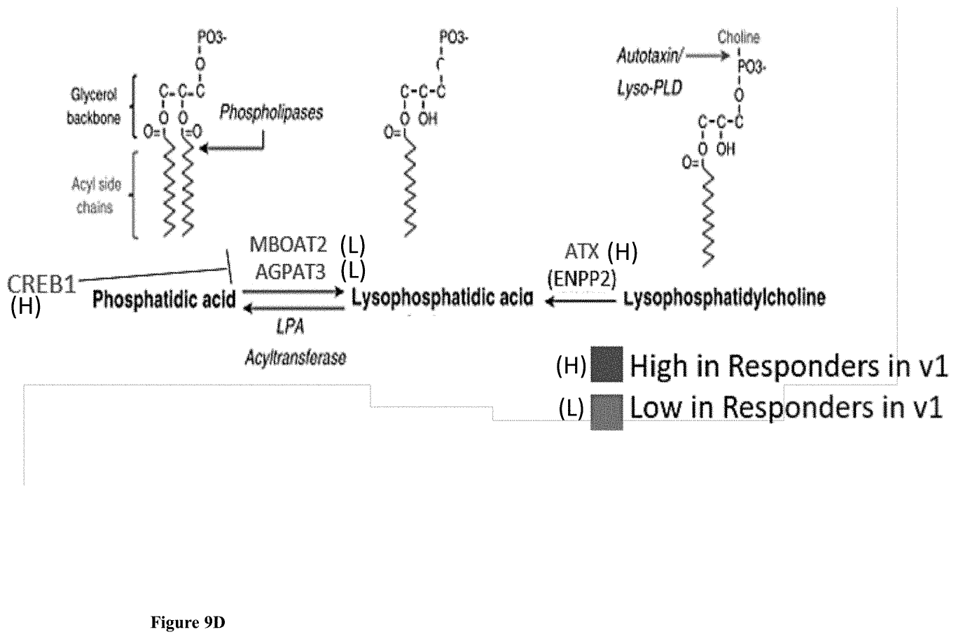

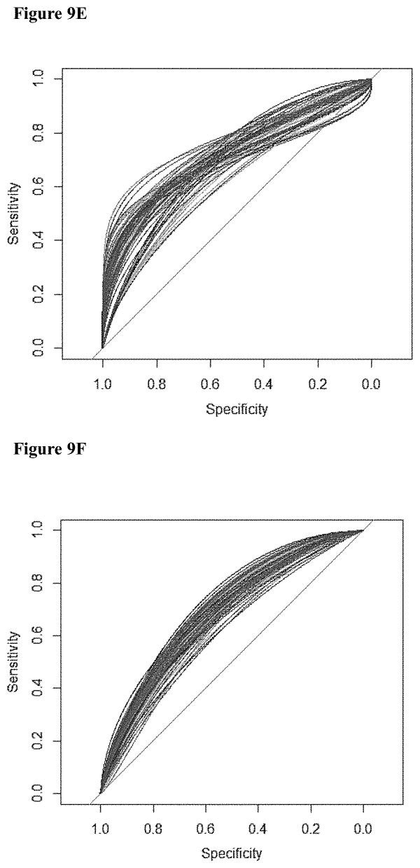

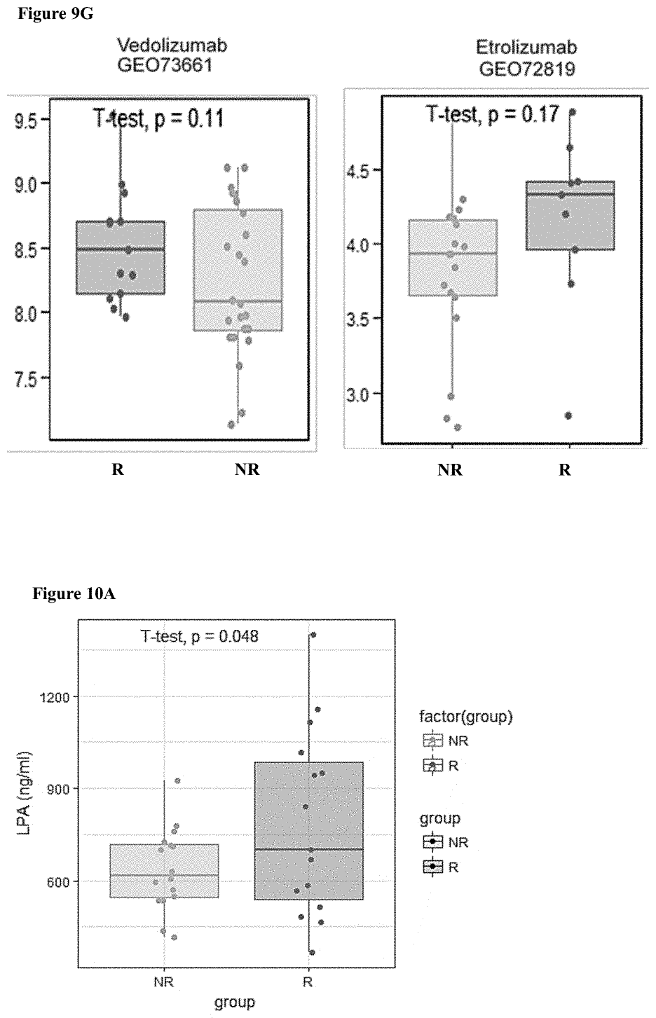

Based on the characterized integrin-related normal drug response, differential expression between responders and non-responders was identified 14 weeks post treatment as shown in FIG. 8A. The interconnected network of those expressed genes is provided in FIG. 8B. Among the detected genes, genes that were also differentially expressed between responders and non-responders at baseline were identified. The analysis revealed 4 genes that were involved in the LPA (Lysophosphatidic acid) metabolism pathway which is highly related to cell migration. Two other genes were also found. The genes related to LPA synthesis were ENPP2 (ATX), MBOAT2, AGPAT3, and CREB1 and the additional genes were SLC22A4 and METTL9 (FIG. 9A).

A second independent validation cohort was tested to confirm the findings related to these 6 genes. As expected ATX and CREB1 were once again upregulated in responders, while AGPAT3, MBOAT2, SLC22A4 and METTL9 were once again downregulated in responders (FIG. 9B).

Logistic regression models examining each gene individually yielded AUC values between 0.7 and 0.8, whereas combining the four LPA-related genes yielded an AUC of 0.93 (95% CI 0.83-1.00) for responsiveness prediction pre-treatment (FIG. 9C). These results suggest that responders present increased LPA synthesis capacity in peripheral blood indicating enhanced migratory potential pre-treatment in blood (FIG. 9D).

When three of the genes, AGPAT3, MBOAT2 and ATX were examined together it was found that also including baseline monocyte abundance in blood improved the predictive value. Abundance of other immune cells (NK, CD4+, etc.) did not improve predictive value. AUC was calculated by performing 100-repeated 10-fold cross validations. Median AUCs of 74.2% [CI 65.0%-79.2%] and 70.0% [CI 66.3.4-73.7] were observed for the primary (FIG. 9E) and validation cohorts (FIG. 9F) respectively when the 3 genes and monocyte abundance was combined.

Interestingly, when expression in the intestines themselves was examined the reverse result was observed. Two public data sets, GE073661 and GE072819, which examined expression before and after Vedolizumab and Etrolizumab treatment respectively, were examined. These data sets recorded expression values in the intestines for non-responders and responders. MBOAT2, an inhibitor of LPA synthesis which was found to be more highly expressed in the blood of non-responders, was more lowly expressed in the intestines of non-responders (FIG. 9G). This suggests there may be an inverse relationship between the expression of LPA regulating genes in the intestines and the blood. It also shows that therapeutic agents geared to converting non-responders to responders, should be different if they are targeting the intestines as opposed to the blood.

### Example 8: Responders Present an Increase in Serum LPA Protein Level

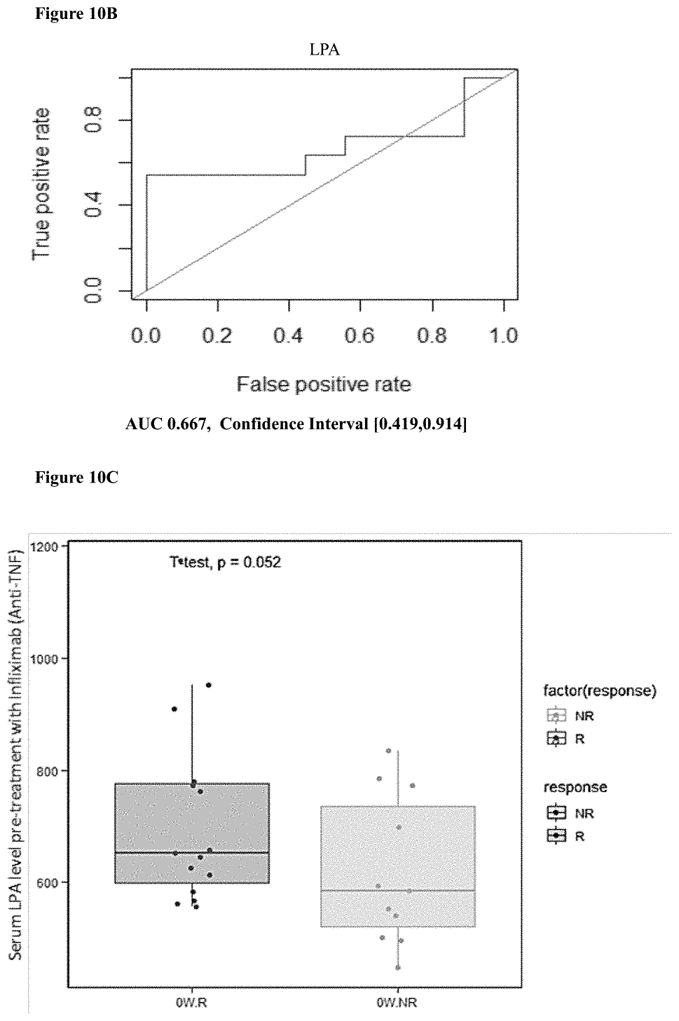

Target validation of the observed genomic findings, at the protein level, was assessed by ELISA using the current patient cohort with extension of additional Vedolizumab treated IBD patients (n=30). As shown in FIG. 10A, serum LPA was increased in responding patients, pre-treatment, compared to non-responders. However, the levels of LPA in the blood stream of patients pre-treatment was less predictive of responsiveness (AUC of 0.667%, CI 0.42-0.91) than each of the four above described genes, and significantly less predictive than all four genes together (FIG. 10B).

### Example 9: Responders to Infliximab Present an Increase in Serum LPA Protein Level

It was next tested in differential expression of the LPA axis may be relevant for additional treatment that exert an effect on local inflammation. The anti-TNFα blocking antibody Infliximab was used to test this hypothesis. ELISA was again used to examine serum LPA levels in Infliximab responding (n=14) and non-responding (n=10) patients before treatment. Response was determined 14 weeks post first treatment. Once again responders had higher serum LPA levels than non-responders pre-treatment (FIG. 10C) indicating that LPA is a biomarker for response to a general class of agents that treat localized inflammation.

### Example 10: Combined Vedolizumab and LPA Decreases Inflammatory Response

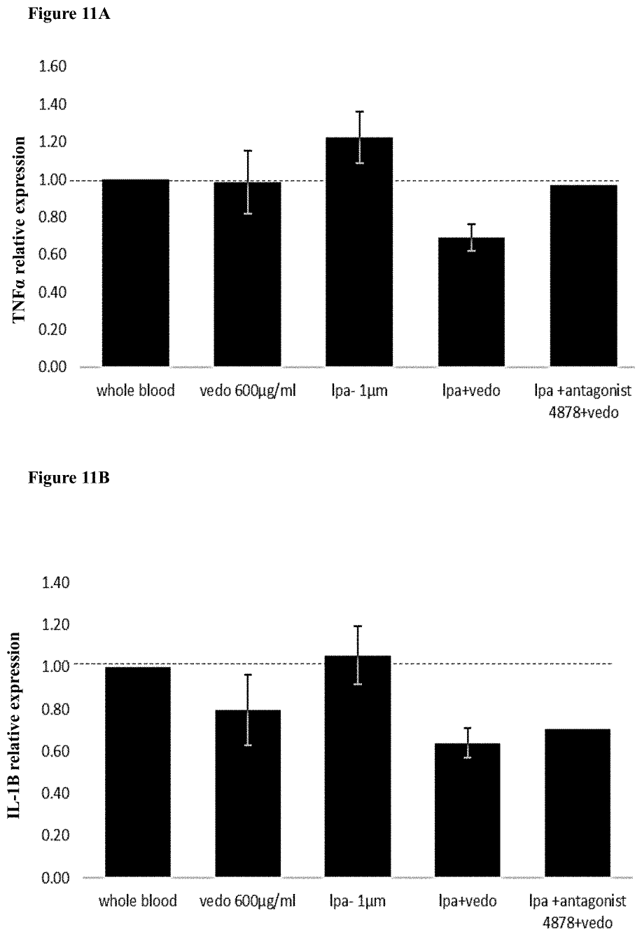

The combined effect of Vedolizumab and LPA was next tested on blood cells. Whole blood from healthy donors was treated with Vedolizumab, LPA or a combination for 3 hours at 37 degrees and pro-inflammatory cytokine expression was measured. Untreated whole blood was used as a control. As can be seen in FIG. 11A, neither Vedolizumab nor LPA alone had an effect on TNFα expression, however, when the two were combined a de novo synergistic effect was observed with cytokine levels decreasing by more than 30%. This effect was specific, as addition of an LPA receptor 2 antagonist abrogated the effect (FIG. 11A). A similar result was observed for IL-1B expression, although in this instance the antagonist was less effective (FIG. 11B). This may be due to the fact that there are 6 LPA receptors and it appears that antagonism of only one of them is not sufficient to completely counter act the effect of the LPA.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining the suitability of a subject in need thereof to be treated with a therapeutic agent that reduces localized inflammation, the method comprising:
a. providing a sample from said subject;
b. measuring in said sample at least one of:
i. expression of lysophosphatidic acid (LPA);
ii. expression of at least one molecule selected from SLC22A4, METTL9, AGPAT3, MBOAT2, ATX and CREB1; and
iii. expression of at least one molecule that regulates LPA expression;
c. determining said suitability of said subject for treatment according to said expression of said LPA, said expression of said at least one molecule or both, wherein expression beyond a predetermined threshold qualifies said subject for treatment with said therapeutic agent and expression within said predetermined threshold disqualifies said subject for treatment with said therapeutic agent; and
d. administering said therapeutic agent that reduces localized inflammation to said suitable subject thereby determining the suitability of a subject to be treated with a therapeutic agent that reduces localized inflammation.

2. (canceled)

3. The method of claim 1, wherein at least one of:
a. the subject suffers from inflammatory bowel disease (IBD);
b. said sample is a peripheral blood sample or a sample from the gut; and
c. said measuring expression comprises measuring mRNA expression, protein expression or both.

4. (canceled)

5. (canceled)

6. The method of claim 1, wherein said at least one molecule that regulates LPA expression upregulates LPA expression, said sample is from peripheral blood and said subject is suitable for treatment if expression of said molecule is above said predetermined threshold or wherein said at least one molecule that regulates LPA expression down-regulates LPA expression, said sample is from peripheral blood and wherein said subject is suitable for treatment if expression of said molecule is below said predetermined threshold.

7. The method of claim 1, wherein said at least one molecule that regulates LPA expression upregulates LPA expression, said sample is a gut sample and said subject is suitable for treatment if expression of said molecule is below said predetermined threshold or wherein said at least one molecule that regulates LPA expression down-regulates LPA expression, said sample is a gut sample and wherein said subject is suitable for treatment if expression of said molecule is above said predetermined threshold.

8. The method of claim 1, wherein said molecule that regulates LPA expression regulates LPA synthesis and is selected from the group consisting of AGPAT3, MBOAT2, ENPP2 (ATX), and CREB1, optionally wherein the said measuring comprises measuring in said sample expression of AGPAT3, MBOAT2, ATX and CREB1.

9. (canceled)

10. The method of claim 1, wherein said subject is suitable for treatment if at least one of:
a. expression in blood of at least one of AGPAT3, SLC22A4, METTL9 and MBOAT2 is below said predetermined threshold, expression in blood of at least one of ATX and CREB1 is above said predetermined threshold, or both; or
b. expression in a gut sample of at least one of AGPAT3, SLC22A4, METTL9 and MBOAT2 is above said predetermined threshold, expression in a gut sample of at least one of ATX and CREB1 is below said predetermined threshold, or both.

11. The method of claim 1, wherein said subject is suitable for treatment if expression of LPA in a gut sample is below said predetermined threshold or said expression of LPA in peripheral blood is above said predetermined threshold.

12. The method of nay any of claim 1, further comprising measuring monocyte abundance in said sample and wherein monocyte numbers below a predetermined threshold is indicative of suitability to be treated.

13. (canceled)

14. The method of claim 1, further comprising inducing a subject unsuitable to be treated with a therapeutic agent that reduces localized inflammation to be suitable to be treated with said therapeutic agent, by increasing LPA levels or activity in said subject, and administering said therapeutic agent that reduces localized inflammation to said subject induced to be suitable.

15. A method of treating a subject unsuitable for treatment with a therapeutic agent that reduces localized inflammation, comprising:
a. increasing LPA levels or activity in said subject, and
b. administering said therapeutic agent that reduces localized inflammation,
thereby treating a subject unsuitable for treatment with a therapeutic agent that reduces localized inflammation.

16. The method of claim 15, wherein increasing LPA levels or function in said subject comprises at least one of:
a. administering an agent that increases LPA levels or function in said subject;
b. increasing LPA levels or function in peripheral blood of said subject;
c. increasing LPA levels or function in peripheral blood and decreasing LPA levels or function in a mucosa of said subject, optionally wherein said mucosa is gut mucosa, said decreasing LPA levels in said gut mucosa of said subject, comprises decreasing expression or activity of at least one molecule that increases LPA levels, increasing expression or activity of at least one molecule that decreases LPA level, blocking LPA binding to a gut LPA receptor, or a combination thereof, or both;
d. increasing expression or activity of at least one molecule that increases LPA levels, decreasing expression or activity of at least one molecule that decreases LPA levels or both, optionally wherein
i. said at least one molecule that increases LPA is ATX, CREB1 or both and wherein said at least one molecule that decreases LPA levels is AGPAT3, MBOAT2, SLC22A4, METTL9 or a combination thereof; or
ii. decreasing activity of at least one molecule that increases LPA levels comprising administering an antagonist or inhibitor of ATX, CREB1 or both and increasing activity of at least one molecule that decreases LPA levels comprises administering an agonist or activator of AGPAT3, MBOAT2, SLC22A4, METTL9 or a combination thereof; or wherein increasing activity of at least one molecule that increases LPA levels comprising administering an agonist or activator of ATX, CREB1 or both and decreasing activity of at least one molecule that decreases LPA levels comprises administering an antagonist or inhibitor of AGPAT3, MBOAT2, SLC22A4, METTL9 or a combination thereof
e. administering to said subject LPA or an LPA precursor; and
f. administering an LPA receptor agonist.

17. (canceled)

18. (canceled)

19. (canceled)

20. (canceled)

21. (canceled)

22. (canceled)

23. (canceled)

24. (canceled)

25. (canceled)

26. (canceled)

27. The method of claim 15, wherein reducing localized inflammation comprises inhibiting cell migration, optionally wherein said cell migration is immune cell migration.

28. (canceled)

29. The method of claim 1, wherein said therapeutic agent is selected from an anti-integrin blocking antibody and an anti-pro-inflammatory cytokine blocking antibody; optionally wherein:
a. said anti-integrin blocking antibody is selected from an anti-ITGA4/B7 blocking antibody, an anti-ITGA4 blocking antibody and an anti-ITGB7 blocking antibody; or
b. said pro-inflammatory cytokine is TNFα.

30. (canceled)

31. The method of claim 29, wherein said anti-integrin blocking antibody is anti-ITGA4/B7 blocking antibody Vedolizumab or anti-ITGB7 blocking antibody Etrolizumab.

32. (canceled)

33. The method of claim 34, wherein said increasing LPA levels or activity in said subject comprises administering LPA to said subject.

34. The method of claim 15, wherein said method is a method of treating inflammation in a subject, the method comprising administering to said subject an anti-integrin blocking antibody and increasing LPA levels or activity in said subject, thereby treating inflammation in a subject.

35. (canceled)

36. A kit comprising:
a. at least 2 detection molecules selected from: a detection molecule specific to ATX, a detection molecule specific to CREB1, a detection molecule specific to AGPAT3, a detection molecule specific to SLC22A4, a detection molecule specific to METTL9 and a detection molecule specific to MBOAT2; or
b. an anti-integrin blocking antibody and an agent that increases LPA levels, function or both.

37. The kit of claim 36, consisting of said detection molecule specific to ATX, said detection molecule specific to CREB1, said detection molecule specific to AGPAT3 and said detection molecule specific to MBOAT2.

38. The kit of claim 36, further comprising a detection molecule specific to LPA.

39. The kit of claim 36, further comprising a therapeutic agent that reduces localized inflammation.

40. (canceled)
